(19) United States
(12) Patent Application Publication
(10) Pub. No.: US 2024/0408179 A1
(43) Pub. Date: Dec. 12, 2024

(54) ORAL DELIVERY OF THERAPEUTIC AGENTS

(71) Applicant: GUANGZHOU DAZHOU BIOMEDICINE LTD., Guangzhou, Guangdong (CN)

(72) Inventors: Steven DINH, Miami, FL (US); Huadong TANG, McLean, VA (US); Wenfei LIANG, Guangzhou, Guangdong (CN)

(21) Appl. No.: 18/702,119

(22) PCT Filed: Oct. 19, 2022
(86) PCT No.: PCT/CN2022/126156

§ 371 Date: Apr. 17, 2024

(30) Foreign Application Priority Data
Oct. 21, 2021 (WO) PCT/CN2021/125360

(51) Int. Cl. A61K 38/26; A61K 9/19; A61K 38/28; A61K 47/12; A61K 47/18

(52) U.S. Cl. CPC A61K 38/26; A61K 9/19; A61K 38/28; A61K 47/12; A61K 47/18

(57) ABSTRACT

Provided herein are pharmaceutical compositions comprising therapeutic agent (s) and functional excipients that can enhance oral bioavailability of the therapeutic agent (s). Also provided herein are methods of preparing the pharmaceutical composition and methods of using the same for treating various diseases or disorders such as type-2 diabetes.

### CROSS REFERENCE TO RELATED APPLICATIONS

This application claims priority of International Application No. PCT/CN2021/125360, filed Oct. 21, 2021, the content of which is incorporated herein by reference in its entirety for all purposes.

### BACKGROUND

### Field of the Invention

In various embodiments, the present invention generally relates to oral delivery of therapeutic agents.

### Background Art

Delivery via oral route is the most preferred for drug administration. Oral route of administration has several advantages with better patient compliance, ease of administration and typically low cost of production, storage and distribution.

For large molecules, however, very few can be administered via the oral route for the following reasons: 1) pre-systemic degradation due to the acidity in the stomach, and enzymes in the gastro intestinal (GI) tract; 2) low absorption across epithelial cells that line absorption surfaces such as those in the GI tract; and 3) post absorption degradation, such as the first pass metabolism.

### BRIEF SUMMARY

In various embodiments, the present disclosure relates to oral delivery of therapeutic agents, in particular, molecules such as those having high molecular weight or otherwise difficult to be absorbed through oral administration, such as polypeptides, etc. The present disclosure is based, in part, on the unexpected discovery that the combination of certain fatty acids and oral absorption enhancers can achieve a synergistic effect in enhancing overall oral absorption of therapeutic agents.

In some embodiments, the present disclosure pertains to the use of mixtures of functional excipients combined with formulation method of preparation to significantly enhance the gastrointestinal absorption of biologic therapeutics as a single agent or combination agents to transform approaches by which diseases are cured and alleviated.

Biologic therapeutics as used herein are not particularly limited, and include carbohydrates, peptides, proteins, enzymes, antibodies, drug conjugates, vaccines, nucleic acids and nucleic acid-based gene therapies. For example, biologic therapeutics include but not limited to unfractionated heparin, low molecular weight heparins, synthetic heparins, growth hormones, growth factors, insulins, insulin icodec, interferons, interlukins, follicular stimulating hormones, gonadotropins, erythropoeitins, incretins, semaglutide, liraglutide, exenatide, tirzepatide, PYY, oxyntomodulin, GLP-1, GLP-2, calcitonin, PTH and analogs, vancomycin, daptomycin, micafungin, anidulafungin, capsofungin, leuprolide, monoclonal antibodies.

Functional excipients useful herein include but not limited to these molecules and their analogs: sodium 8-(2-hydroxybenzamido)octanoate (SNAC), 10-((2-hydroxybenzoyl)amino)decanoate sodium (SNAD), 8-(N-2-hydroxy-5-chlorobenzoyl)-amino-caprylates (5CNA C), sodium N-(4-chlorosalicyloyl)-4-aminobutyrate (4-CNAB), sodium N-[8-(2-hydroxy-4-methoxy)bensoyl]amino caprylate (4-MOAC), Bis-3,6(4-fumarylaminobutyl)-2,5-diketopiperazirne. In addition, functional excipients useful herein include, for example, linear fatty acids and their salts with the number of carbons in the aliphatic chain ranging from 2 to 20.

In certain illustrative embodiments, the medication is administered using oral dosage forms that contain an active agent of incretin therapeutics (“incretins”) such as GLP-1 receptor agonists (GLP-1 RA), functional excipients such as mixtures of fatty acids and surfactants, and common excipients used in oral dosage forms such as tablets and capsules. Functional excipients useful herein include but not limited to sodium N-[8(-2-hydroxybenzoyl)amino]caprylate (SNAC), 8-(N-2-hydroxy-5-chlorobenzoyl)-amino-caprylic acid (5-CNAC), [Bis-3,6(4-fumarylaminobutyl)-2,5-diketopiperazine, salts of linear fatty acids, and combinations thereof. In other embodiments, the oral dosage forms can contain combination of active agents such as but not limited to GLP-1 RA and SGLT-2 inhibitors, GLP-1R-A and DPP4 inhibitors, and GLP-1 RA and insulin. Incretins useful for embodiments herein include but not limited GLP-1, GIP, GLP-1/GIP agonists. Incretins useful for embodiments herein also include GIP, GLP-1/GIP agonist in clinical trials, GLP-1 RA and GLP-1 analogues including but not limited to semaglutide, liraglutide, dulaglutide, lixisenatide, exenatide and others. SGLT2 inhibitors useful for embodiments herein include but not limited to empagliflozin, canagliflozin, dapagliflozin, ertugliflozin and others. DPP4 inhibitors useful for embodiments herein include but not limited to sitagliptin, vildagliptin, saxagliptin, linagliptin, alogliptin, and others. Insulin and insulin analogues useful for embodiments herein include but not limited to insulin icodec. In yet other embodiments, the present disclosure pertains to methods for the preparation of oral dosage forms such as tablets and capsules. In further embodiments, mixtures of functional excipients and methods of preparation described herein can also be applied to other routes of administration.

Exemplary embodiments of the present disclosure are also shown in claims 1-43 as described herein.

It is to be understood that both the foregoing summary and the following detailed description are exemplary and explanatory only, and are not restrictive of the invention herein.

### BRIEF DESCRIPTION OF THE FIGURES

FIG. 1 shows a comparison of mean pharmacokinetics of semaglutide in plasma in a linear scale following oral administration of (1) formulations containing 10 mg semaglutide with 300 mg SNAC alone or (2) formulations containing 10 mg semaglutide, 300 mg SNAC, and 150 mg sodium caprate. Error bars indicate the standard deviations of concentrations of semaglutide at specified times.

FIG. 2 presents a graph showing semaglutide concentration in plasma in a log-linear scale over time profile following (1) oral administration of 10 mg of semaglutide and 300 mg SNAC; or (2) oral administration of 10 mg of semaglutide, 300 mg SNAC, and 150 mg of sodium caprate. Error bars indicate the standard deviations of concentrations of semaglutide at specified times.

FIG. 3 shows glucose concentration over time profile of the oral glucose tests in healthy Beagle dogs, following (1) negative control, (2) oral administration of 10 mg of semaglutide and 300 mg SNAC; or (3) oral administration of 10 mg of semaglutide, 300 mg SNAC, and 150 mg of sodium caprate.

FIG. 4 shows presents a graph showing semaglutide concentration in plasma over time profile comparing oral administration of 7 mg semaglutide formulated in 300 mg SNAC/150 mg C10 by freeze-drying to that of 7 mg semaglutide of a commercial tablet (Rybelsus).

FIG. 5 shows presents a graph showing semaglutide concentration in plasma over time profile comparing oral administration of 10 mg semaglutide formulated in 300 mg SNAC/150 mg C10 by freeze-drying versus by simple blending.

FIG. 6 shows presents a graph showing semaglutide concentration in plasma over time profile comparing oral administration of 10 mg semaglutide formulated in 300 mg SNAC, 300 mg C10, or 300 mg SNAC/150 mg C10, each prepared by freeze-drying method.

### DETAILED DESCRIPTION

The present disclosure generally relates to oral delivery of therapeutic agents. As detailed herein, the present inventors have discovered that the combination of semaglutide with SNAC and sodium caprate produced an unexpected higher drug absorption and more effective glucose control than formulations containing semaglutide and SNAC alone. The effective enhancement based on semaglutide plasma concentration is about 5-fold. The inter-individual variability is also substantially reduced. In addition, the superior pharmacological effect of semaglutide based on oral glucose tolerance test (oGTT) was also demonstrated for the combination of semaglutide with SNAC and sodium caprate. Additionally, the data here shows that the use of freeze-drying method in preparing the formulation herein is beneficial in achieving a higher exposure of semaglutide compared to equivalent formulation prepared by using a simple blend method. Further, the data here establishes that the use of SNAC and sodium caprate achieved a synergistic effect in enhancing oral exposure of semaglutide.

In a broad aspect, the present disclosure provides a pharmaceutical composition comprising a therapeutic agent (e.g., any of those described herein) and one or more, particularly, two or more, functional excipients (e.g., any of those described herein). Unless otherwise contrary from context, functional excipients as used herein refer to those excipients that can enhance the oral bioavailability of the therapeutic agent. For example, in some embodiments, the functional excipients refer to those that can increase the bioavailability of the GLP-1 agonist of a composition following oral administration.

Typically, the pharmaceutical composition comprises two or more functional excipients that can synergistically enhance the oral absorption of the therapeutic agent. In some embodiments, the one or more functional excipients include an aliphatic acid of Formula I: RCOOH, wherein R represents an aliphatic group, or a pharmaceutically acceptable salt thereof. In some embodiments, the one or more functional excipients include a compound of Formula II:

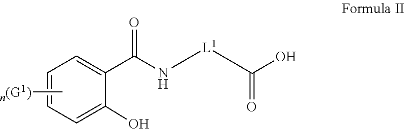

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene. Typically, the pharmaceutical composition is formulated for oral administration. Preferably, a therapeutically effective plasma concentration of the therapeutic agent can be achieved following oral administration of the pharmaceutical composition herein.

In some embodiments, the present disclosure provides a pharmaceutical composition comprises (a) a therapeutic agent (e.g., any of those described herein), (b) an aliphatic acid of Formula I: RCOOH, wherein R represents an aliphatic group, or a pharmaceutically acceptable salt thereof, and (c) a compound of Formula II:

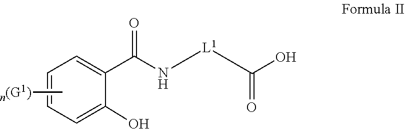

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

### Therapeutic Agents

The therapeutic agent useful for the pharmaceutical compositions described herein is not particularly limited. For example, the therapeutic agent can include a carbohydrate, peptide, protein, antibody, vaccine, nucleic acid, etc. In some embodiments, the therapeutic agent can be a Biologic therapeutics as described herein. In some embodiments, the therapeutic agent can be a large molecule, for example, those having a molecular weight of more than 2,000 Daltons, more than 3,000 Daltons, more than 10,000 Daltons, or more than 100,000 Daltons, etc.

For example, in some embodiments, the therapeutic agent can be a carbohydrate, such as a heparin (e.g., unfractionated heparin, Low molecular weight heparins, Synthetic heparins such as Fondaparinux) or glucosamines, etc.

In some embodiments, the therapeutic agent can be a polypeptide (alternatively referred to herein as peptide), including proteins and antibodies. Useful polypeptides for embodiments herein are not particularly limited and include for example, the following agents:
1. GLP-1 and analogs
   a) GLP-1
   b) Semaglutide
   c) Liraglutide
   d) Exenatide
   e) Tirzepatide
   f) Lixisenatide
   g) Efinopegdutide
   h) Cotadutide
2. Teduglutide
3. Pramlintide
4. PYY
5. Oxvntomodulin
6. Glucagon
7. Calcitonin
8. Octreotide
9. PTI and analogs
   a) Teriparatide
10. Etelcalcetide
11. Oxytocin
12. Antibiotics
   a) Vancomycin
   b) Daptomycin
   c) Dalbavancin
   d) Oritavancin
   e) Telavancin
13. Antifungals
   a) Micafungin
   b) Anidulafungin
   c) Capsofungin
14. Vasopressin
15. Leuprolide
16. Nesiritide
17. Enfivirtide
18. Growvth hormones and analogs
19. Pegvisonant
20. Mecasermin (rhIGF-1)
21. GnRH
   a) Hlistrelin
22. Dibotermin-ca
23. Palifermin
24. Becaplennin2
25. Adrenocorticotropin
26. Somatostatin
27. Oxodotreotide
28. Pasireotide
29. Trypsin
30. Insulin and analogs
   a) Insulin
   b) Insulin icodec
   c) Insulin deteinir
   d) Insulin glargine
   e) Insulin lispro
   f) Insulin glulisine
   g) Insulin aspart
31. Interferons
   a) Interferon alfacon 1
   b) Interferon-α2a
   c) Interferon-α2b
   d) Interferon-αn3
   e) Interferon-β1a
   f) Interferon-μ1b
   g) Interferon-γ1b
32. Interlukins
   a) Oprelvekin
   h) Aldesleukin
   i) Denileukin
33. Follicular stimulating hormone
34. Human chorionic gonadotropin
35. Lutropin-α
36. Erythropoietin
37. Epoetin-α
38. Darbepoetin-α
39. Filgrastim
40. Pegfilgrastim
41. Sargramostim
42. Alteplase
43. Reteplase
44. Tenecteplase
45. Uirokinase
46. Factor VIIa
47. Factor Vi
48. Factor LX
49. Antithrombin IIH
50. Protein C
51. Drotrecogin-α
52, β-Glucocerebrosidase
53. Alglucosidase-α
54. Laronidase
55. Idursulphase
56. Galsulphase
57. Agalsidase-β
58. α-1-Proteinase inhibitor
59. Lactase2
60. Pancreatic enzymes
61. Adenosine deaminase
62. Immunoglobulins
63. Albumin
64. Lepirudin
65. Bivalirudin
66. Streptokinase
67. Anistreplase
68. Crizanlizumab
69. Etanercept
70. Adalimumab
71. Infliximab
72. Bevacizumab
73. Trastuzumab
74. Rituximab
75. Copaxone
76. Patisiran
77. Givosiran
78. Lumasiran

In some embodiments, the therapeutic agent can also include a vaccine. In some embodiments, the therapeutic agent can also include a nucleic acid.

In some preferred embodiments, the therapeutic agent can include an incretin therapeutics. For example, in some preferred embodiments, the therapeutic agent can include a Glucagon-Like Peptide-1 (GLP-1) receptor agonist, e.g., any of those described herein, or any of those described in U.S. Pat. Nos. 10,960,052, 8,129,343, 8,536,122, 9,278,123, 10,086,047, 10,278,923, and 10,933,120, the entire contents of each of which are herein incorporated by reference. In some preferred embodiments, the therapeutic agent can include semaglutide, liraglutide, dulaglutide, lixisenatide, or exenatide. Other incretins and peptides can include, but not limited to, PYY and PYY analogues; GLP-1/GIP receptor due agonists such as, but not limited to Tirzepatide, CT-388, SCO-094, etc.; GLP-1/GCGR receptor due agonists, such as, but not limited to efinopegdutide, IB1362, etc.

In some preferred embodiments, the pharmaceutical composition herein can include the GLP-1 receptor agonist as the only therapeutic agent. In some embodiments, the pharmaceutical composition herein can include the GLP-1 receptor agonist as one therapeutic agent in combination with one or more other therapeutic agent that is useful for treating diabetes, e.g., any of those known in the art. In some embodiments, the one or more other therapeutic agent can include (1) a SGLT-2 inhibitor, such as empagliflozin, canagliflozin, dapagliflozin, or ertugliflozin; (2) a DPP-4 inhibitor, such as sitagliptin, vildagliptin, saxagliptin, linagliptin, or alogliptin; (3) insulin or insulin analogues (e.g., Insulin icodec); (4) GIP, glucose-dependent insulinotropic polypeptides; and/or (5) amylin or amylin analogues. In some embodiments, the one or more other therapeutic agent can also include (1) biguanides; (2) Thiazolidinediones; (3) DPP-4 inhibitors; (4) PYY; and (5) sulfonylureas.

In some embodiments, the pharmaceutical composition herein can include the GLP-1 receptor agonist as one therapeutic agent in combination with one or more other therapeutic agent that is useful for treating a neurological disease, such as Alzheimer's Disease. For example, in some embodiments, the pharmaceutical composition herein can include semaglutide as one therapeutic agent in combination with (1) Cholinesterase inhibitors (e.g., Aricept, Exelon, Razadyne); (2) Glutamate regulators (e.g., Namenda); and/or (3) Orexin receptor antagonist (e.g., Belsomra).

In more preferred embodiments, the therapeutic agent herein can include semaglutide. Semaglutide as used herein is not limited to any particular forms. For example, in some embodiments, semaglutide can be in the form of a pharmaceutically acceptable salt, such as a sodium salt. Semaglutide is marketed in the United States under several brandnames, including the oral Rybelsus® tablet formulation. See Rybelsus Prescribing Information approved by the U.S. Food and Drug Administration, 2021 version, the content of which is herein incorporated by reference in its entirety. As described therein, the peptide backbone of semaglutide is produced by yeast fermentation. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by the enzyme dipeptidyl-peptidase 4 (DPP-4). A minor modification was made in position 34 to ensure the attachment of only one fatty di-acid. The molecular formula is C1871291N45059 and the molecular weight is 4113.58 g/mol. The structure is shown below:

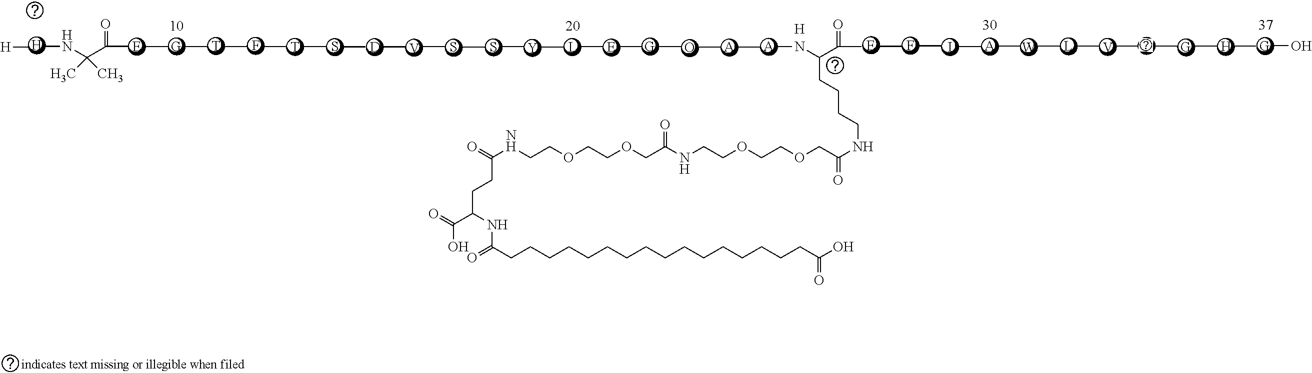

In some preferred embodiments, the pharmaceutical composition herein can include semaglutide as the only therapeutic agent. In some embodiments, the pharmaceutical composition herein can include semaglutide as one therapeutic agent in combination with one or more other therapeutic agent that is useful for treating diabetes, e.g., any of those known in the art. In some embodiments, the one or more other therapeutic agent can include (1) a SGLT-2 inhibitor, such as empagliflozin, canagliflozin, dapagliflozin, or ertugliflozin; (2) a DPP-4 inhibitor, such as sitagliptin, vildagliptin, saxagliptin, linagliptin, or alogliptin; (3) insulin or insulin analogue (e.g., Insulin icodec); (4) GIP, glucose-dependent insulinotropic polypeptides; and/or (5) amylin or amylin analogues. In some embodiments, the one or more other therapeutic agent can also include (1) biguanides; (2) Thiazolidinediones; (3) DPP-4 inhibitors; (4) PYY; and (5) sulfonylureas. In some embodiments, the one or more other therapeutic agent can include one or more selected from the following: Biguanides, Sulfonylureas and meglitinides, Thiazolidinediones, Alpha-glucosidase inhibitors, other Glucagon like peptide-1 (GLP-1) receptor agonists. Dipeptidyl peptidase 4 (DPP4) inhibitors, Amylin analogue, Sodium-glucose cotransporter 2 (SGLT-2) inhibitors, Dopamine agonists, and Bile acid sequestrants. In some embodiments, the one or more other therapeutic agent can include one or more selected from the following: Metformin, Glipizide, Gliclazide, Glyburide, Glimepiride, Nateglinide, Repaglinide, Pioglitazone, Rosiglitazone, Acarbose, Miglitol, Voglibose, Exenatide, Liraglutide, Lixisenatide, Dulaglutide, Albiglutide, Sitagliptin, Vildagliptin, Saxagliptin, Linagliptin, Gemigliptin, Anagliptin, Teneligliptin, Alogliptin, Trelagliptin, Omarigliptin, Evogliptin, Gosogliptin, Pramlintide, Canagliflozin, Dapagliflozin, Empagliflozin. Ipragliflozin, Bromocriptine, and Colesevelam.

In some embodiments, the pharmaceutical composition herein can include semaglutide as one therapeutic agent in combination with one or more other therapeutic agent that is useful for treating a neurological disease, such as Alzheimer's Disease. For example, in some embodiments, the pharmaceutical composition herein can include semaglutide as one therapeutic agent in combination with (1) Cholinesterase inhibitors (e.g., Aricept, Exelon, Razadyne); (2) Glutamate regulators (e.g., Namenda); and/or (3) Orexin receptor antagonist (e.g., Belsomra).

### Aliphatic Acid of Formula I

Typically, the pharmaceutical composition herein comprises an aliphatic acid of Formula I: RCOOH, wherein R represents an aliphatic group, or a pharmaceutically acceptable salt thereof.

Useful aliphatic acids are not particularly limited. For example, in some embodiments, the aliphatic acid has a Formula I: RCOOH, wherein R represents an alkyl group having 1-30 carbon atoms. The alkyl group can be a linear or branched chain alkyl group. For example, in some embodiments, R in Formula I can be —(CH₂)₁₋₁₈CH₃. In some embodiments, R in Formula I can be an alkyl group having 3-20 carbon atoms. In some embodiments, R in Formula I can be an alkyl group having 5-16 carbon atoms. In some embodiments, the aliphatic acid of Formula I is a linear aliphatic acid having 2 to 20 carbon atoms, such as caprylic acid, capric acid, or lauric acid. In any of the embodiments described herein, unless otherwise specified or contrary from context, the aliphatic acid of Formula I can be capric acid.

The aliphatic acid of Formula I can be present in the pharmaceutical composition herein as a free acid or any pharmaceutically acceptable salt thereof, such as an alkali or alkaline salt thereof, for example, a sodium or potassium salt. In some preferred embodiments, the pharmaceutical composition herein comprises sodium caprate.

### Compound of Formula II

Typically, the pharmaceutical composition herein comprises a compound of Formula II:

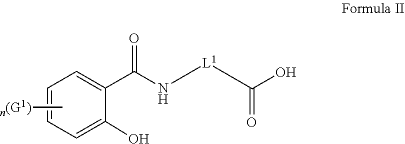

or a pharmaceutically acceptable salt thereof, wherein the variables are defined herein.

In some embodiments, the compound of Formula II can have no G¹ substituents on the phenyl ring, i.e., n is 0.

In some embodiments, the compound of Formula II can have one G¹ substituted on the phenyl ring, i.e., n is 1. In some embodiments, in Formula II, n is 1, and G is a halogen, C₁₋₄ alkyl, or C₁₋₄ alkoxy. In some embodiments, in Formula II, n is 1, and G¹ is Cl. In some embodiments, in Formula II, n is 1, and G¹ is OCH₃.

L¹ in Formula II is typically a substituted or unsubstituted C₂-C₁₆ alkylene. For example, in some embodiments, L¹ is an unsubstituted C₃-C₁₅ alkylene. In some embodiments, L¹ is an unsubstituted C₅-C₁₃ alkylene. The alkyelene can be a straight-chained or a branched alkyelene. For example, in some embodiments, L¹ is an unsubstituted, straight-chained C₅-C₉ alkylene.

In some preferred embodiments, the compound of Formula II can be

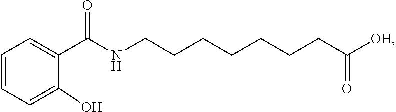 [SMILES: O=C(O)CCCCCCCNC(=O)c1ccccc1O]

which has a chemical name of 8-(2-hydroxybenzamido)octanoic acid (ChemDraw Software, version 20.0). In preferred embodiments, the pharmaceutical composition herein comprises a salt (preferably sodium salt) of 8-(2-hydroxybenzamido)octanoic acid, which can be prepared using the method described in e.g. WO96/030036, WO00/046182, WO01/092206 or WO2008/028859. The salt of 8-(2-hydroxybenzamido)octanoic acid (alternatively known as N-(8-(2-hydroxybenzoyl)amino)caprylic acid) may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino) caprylic acid as well as combinations thereof. In some embodiments, the pharmaceutical composition herein comprises a salt of N-(8-(2-hydroxybenzoyl)amino)caprylic acid as described in WO2007/121318.

In more preferred embodiments, the pharmaceutical composition comprises sodium N-(8-(2-hydroxybenzoyl)amino)caprylate (referred to as “SNAC” herein).

In some preferred embodiments, the compound of Formula II can be

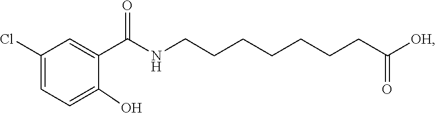 [SMILES: O=C(O)CCCCCCCNC(=O)c1cc(Cl)ccc1O]

which has a chemical name of 8-(5-chloro-2-hydroxybenzamido)octanoic acid (ChemDraw Software, version 20.0). In some embodiments, the pharmaceutical composition herein comprises a salt of 8-(5-chloro-2-hydroxybenzamido)octanoic acid.

In some embodiments, the compound of Formula II can be

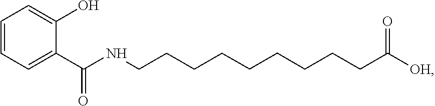 [SMILES: O=C(O)CCCCCCCCCNC(=O)c1ccccc1O]

which has a chemical name 10-((2-hydroxybenzoyl)amino)decanoic acid. In some embodiments, the pharmaceutical composition herein comprises a salt of 10-((2-hydroxybenzoyl)amino)decanoic acid, such as sodium 10-((2-hydroxybenzoyl)amino)decanoate.

In some embodiments, the compound of Formula II can be

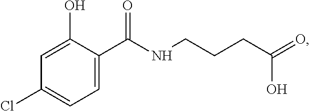 [SMILES: O=C(O)CCCNC(=O)c1ccc(Cl)cc1O]

which has a chemical name N-(4-chlorosalicyloyl)-4-aminobutyric acid. In some embodiments, the pharmaceutical composition herein comprises a salt of N-(4-chlorosalicyloyl)-4-aminobutyric acid, such as sodium N-(4-chlorosalicyloyl)-4-aminobutyrate.

In some embodiments, the compound of Formula II can be

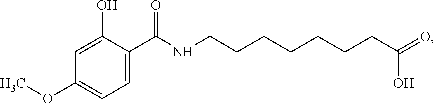 [SMILES: COc1ccc(C(=O)NCCCCCCCC(=O)O)c(O)c1]

which has a chemical name N-[8-(2-hydroxy-4-methoxy)benzoyl]amino caprylic acid. In some embodiments, the pharmaceutical composition herein comprises a salt of N-[8-(2-hydroxy-4-methoxy)benzoyl]amino caprylic acid, such as sodium N-[8-(2-hydroxy-4-methoxy)benzoyl]amino caprylate.

The combinations of therapeutic agent, aliphatic acid of Formula I and the compound of Formula II are not particularly limited.

In some preferred embodiments, the pharmaceutical composition herein comprises (a) a polypeptide (e.g., any of those described herein, such as semaglutide); (b) capric acid or a pharmaceutically acceptable salt thereof; and (c) the compound of Formula II or a pharmaceutically acceptable salt thereof.

In some preferred embodiments, the pharmaceutical composition herein comprises (a) a polypeptide (e.g., any of those described herein, such as semaglutide); (b) a linear aliphatic acid having 2 to 20 carbon atoms, such as caprylic acid, capric acid, or lauric acid or a pharmaceutically acceptable salt thereof; and (c) SNAC.

In some preferred embodiments, the pharmaceutical composition herein comprises (a) a polypeptide (e.g., any of those described herein, such as semaglutide); (b) capric acid or a pharmaceutically acceptable salt thereof; and (c) 8-(2-hydroxybenzamido)octanoic acid or a pharmaceutically acceptable salt thereof.

In some preferred embodiments, the pharmaceutical composition herein comprises (a) a polypeptide (e.g., any of those described herein, such as semaglutide); (b) sodium caprate; and (c) SNAC.

In some preferred embodiments, the pharmaceutical composition herein comprises (a) semaglutide; (b) sodium caprate; and (c) SNAC.

Typically, in the pharmaceutical compositions herein, as applicable, the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, such as about 3:1, about 2:1, about 1:1, about 1:1.5, about 1:2, about 1:2.5, or about 1:3, or any ranges between the recited values, e.g., about 1:2. For example, in some embodiments, the pharmaceutical composition herein comprises (a) semaglutide; (b) sodium caprate; and (c) SNAC, wherein the weight ratio of sodium caprate to SNAC is about 5:1 to about 1:5, such as about 3:1, about 2:1, about 1:1, about 1:1.5, about 1:2, about 1:2.5, or about 1:3, or any ranges between the recited values.

Typically, for each unit dosage form, the combined amount of (b) the aliphatic acid of Formula I or pharmaceutically acceptable salt thereof and (c) the compound of Formula II or pharmaceutically acceptable salt thereof ranges from about 100 mg to about 900 mg, such as about 300 mg, about 450 mg, about 600 mg, about 750 mg, or about 900 mg, or any ranges or values between the recited values. For example, in some embodiments, a unit dosage form of the pharmaceutical composition herein, such as a tablet or capsule, can contain (a) a polypeptide (e.g., any of those described herein, such as semaglutide); (b) sodium caprate and (c) SNAC, wherein the combined amount of sodium caprate and SNAC ranges about 300-600 mg, such as about 450 mg. The amount of the therapeutic agent (e.g., a polypeptide herein such as semaglutide) for each unit dosage form is not particularly limited, for example, typically, the therapeutic agent can be in an amount of about 1 mg to about 200 mg (e.g., about 10 mg, about 50 mg, about 100 mg, or any range between the recited values).

In some embodiments, the pharmaceutical composition comprises the aliphatic acid of Formula I or pharmaceutically acceptable salt thereof in an amount of about 50 mg to about 300 mg per unit dose, such as about 50 mg, about 100 mg, about 150 mg, about 200 mg, about 300 mg, or any range between the recited value, per unit dose. As used herein, unless otherwise specified or obviously contrary from context, the weight of the aliphatic acid of Formula I or pharmaceutically acceptable salt thereof should be understood as the equivalent weight expressed as the weight of the free acid. However, when referring to the amount of sodium caprate specifically, the amount should be understood as the weight of the sodium salt itself, not the corresponding equivalent weight of capric acid. In some embodiments, the pharmaceutical composition comprises the aliphatic acid of Formula I or pharmaceutically acceptable salt thereof in an amount of at least 0.6 mmol (millimole), such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol, per unit dose. In some embodiments, the pharmaceutical composition comprises the aliphatic acid of Formula I or pharmaceutically acceptable salt thereof in an amount of 0.6 mmol to 2 mmol, such as 0.8 mmol to 1.3 mmol, 0.9 mmol to 1.1 mmol, such as 0.95 mmol, 1.0 mmol, etc., per unit dose.

In some embodiments, the pharmaceutical composition comprises the compound of Formula II or pharmaceutically acceptable salt thereof in an amount of about 200 mg to about 400 mg per unit dose, such as about 200 mg, about 250 mg, about 300 mg, about 350 mg, about 400 mg, or any range between the recited value, per unit dose. As used herein, unless otherwise specified or obviously contrary from context, the weight of the compound of Formula II or pharmaceutically acceptable salt thereof should be understood as equivalent weight expressed as the weight of the compound of Formula II. However, when referring to the amount of SNAC specifically, the amount should be understood as the weight of the sodium salt itself, not the corresponding equivalent weight of the acid. In some embodiments, the pharmaceutical composition comprises the compound of Formula II or pharmaceutically acceptable salt thereof in an amount of at least 0.6 mmol, such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol, per unit dose. In some embodiments, the pharmaceutical composition comprises the compound of Formula II or pharmaceutically acceptable salt thereof in an amount of 0.6 mmol to 2 mmol, such as 0.8 mmol to 1.3 mmol, or 0.9 mmol to 1.1 mmol, such as 1 mmol, per unit dose.

Typically, the pharmaceutical composition comprises a synergistic combination of (b) the aliphatic acid of Formula I or pharmaceutically acceptable salt thereof and (c) the compound of Formula II or pharmaceutically acceptable salt thereof, for achieving enhanced oral delivery of the therapeutic agent, such as the polypeptide.

In some specific embodiments, the pharmaceutical composition comprises (a) a therapeutic agent (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). The amount of the therapeutic agent is not particularly limited, for example, typically, the therapeutic agent can be in an amount of about 1 mg to about 200 mg (e.g., about 10 mg, about 50 mug, about 100 mg, or any range between the recited values).

In some specific embodiments, the pharmaceutical composition comprises (a) a therapeutic agent (e.g., any of those described herein); (b) sodium caprate in an amount of about 0.6 mmol to 2 mmol (e.g., 0.6 mmol 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of the therapeutic agent is not particularly limited, for example, typically, the therapeutic agent can be in an amount of about 1 mg to about 200 mg (e.g., about 10 mg, about 50 mg, about 100 mug, or any range between the recited values) or about 0.1 micromole to about 2 micromole.

In some specific embodiments, the pharmaceutical composition comprises (a) a polypeptide (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). The amount of the polypeptide is not particularly limited, for example, typically, the polypeptide can be in an amount of about 1 mg to about 200 mg (e.g., about 10 mg, about 50 mg, about 100 mg, or any range between the recited values).

In some specific embodiments, the pharmaceutical composition comprises (a) a polypeptide (e.g., any of those described herein); (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mol, 1.1 mmol, 1.3 mmol, 2 mmol, or any range between the recited values, such as 0.9-1.3 mmol). The amount of the polypeptide is not particularly limited, for example, typically, the polypeptide can be in an amount of about 1 mg to about 200 mg (e.g., about 10 mg, about 50 mg, about 100 mg, or any range between the recited values).

In some specific embodiments, the pharmaceutical composition comprises (a) a GLP-1 agonist (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mug, about 400 mg, or any range between the recited values). The amount of the GLP-1 agonist is not particularly limited, for example, typically, the GLP-1 agonist can be in an amount of about 1 mg to about 200 ng (e.g., about 10 mg, about 50 mg, about 100 mg, or any range between the recited values.

In some specific embodiments, the pharmaceutical composition comprises (a) a GLP-1 agonist (e.g., any of those described herein); (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of the GLP-1 agonist is not particularly limited, for example, typically, the GLP-1 agonist can be in an amount of about 1 mg to about 200 mg (e.g., about 10 mg, about 50 mg, about 100 mg, or any range between the recited values.

In some specific embodiments, the pharmaceutical composition comprises (a) semaglutide; (b) sodium caprate in an amount of about 50 mg to about 300 mg (e.g., about 100 mg, about 150 mg, about 200 rug, or any range between the recited values); and (c) SNAC in an amount of about 200 mg to about 400 mg (e.g., about 200 mg, about 300 rug, about 400 mg, or any range between the recited values). The amount of semaglutide is not particularly limited, for example, typically, the semaglutide can be in an amount of about 1 mg to about 200 mg (e.g., about 10 mg, about 50 rug, about 100 rug, or any range between the recited values. In some embodiments, the semaglutide can be in an amount of about 0.1 micromole to about 2.5 micromole, such as about 0.5 micromole to about 2.5 micromole.

In some specific embodiments, the pharmaceutical composition comprises (a) semaglutide; (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of semaglutide is not particularly limited, for example, typically, the semaglutide can be in an amount of about 1 mg to about 200 mg (e.g., about 10 mg, about 50 mg, about 100 mg, or any range between the recited values. In some embodiments, the semaglutide can be in an amount of about 0.1 micromole to about 2.5 micromole, such as about 0.5 micromole to about 2.5 micromole.

In any of the embodiments described herein, unless otherwise specified or contrary from context, the pharmaceutical composition can be in the form of a solid oral dosage form. For example, the pharmaceutical composition herein can typically be a capsule or tablet. In some embodiments, the pharmaceutical composition herein can be presented in discrete units (which is referred to herein as “unit dosage forms” or “dosage units”), such as capsules, pills, cachets, lozenges, or tablets, each containing a predetermined amount of the active compound(s). In any of the embodiments described herein, unless otherwise specified or contrary from context, the pharmaceutical composition can be in a unit dosage form. To be clear, the pharmaceutical composition herein can include one or more dosage units. For example, the pharmaceutical composition herein can typically be a capsule or tablet, wherein each capsule or tablet constitutes a dosage unit. As used herein, each “unit dose” of the pharmaceutical composition refers to the dose of the pharmaceutical composition for each administration, which may contain one or more unit dosage forms or dosage units; when more than one dosage units are used to satisfy the unit dose, the dosage units can be the same or different. In preferred embodiments, each unit dose contains a single dosage unit.

The pharmaceutical composition herein can optionally include one or more further excipients, such as those suitable for oral administration. For example, in some embodiments, the pharmaceutical composition herein includes at least one pharmaceutically acceptable excipient. The term “excipient” as used herein broadly refers to any component other than the active therapeutic ingredient(s). The excipient may be an inert substance, an inactive substance, and/or a not medicinally active substance. The excipient may serve various purposes, e.g. as a carrier, vehicle, filler, binder, lubricant, glidant, disintegrant, flow control agents, crystallization retarders, solubilizers, stabilizer, colouring agent, flavouring agent, surfactant, enzyme inhibitors, basifiers, acidifiers, emulsifier and/or to improve administration, and/or absorption of the active substance, tablet coating agents to control the dissolution rates of the solid dosage form according to the pH in the GI tract. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005). In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, tale, etc.; crystallization retarders such as Povidone, etc.; solubilizers such as Pluronic, Povidone, etc.; colouring agents, including dyes and pigments such as Iron Oxide Red or Yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In some embodiments, the pharmaceutical composition herein can comprise a lubricant, a binder, a filler, and/or a chelating agent (e.g., ethylene diamine tetraacetate (EDTA)). However, in some embodiments, the pharmaceutical composition herein can also be free or substantially free of a lubricant, such as having less than 0.1% by weight, less than 0.05% by weight, less than 0.01% by weight, or non-detectable amount, of a lubricant. In some embodiments, the pharmaceutical composition herein can be free of magnesium stearate. In some embodiments, the pharmaceutical composition herein can also be free or substantially free of a binder, such as having less than 0.1% by weight, less than 0.05% by weight, less than 0.01% by weight, or non-detectable amount, of a binder. In some embodiments, the pharmaceutical composition herein can also be free or substantially free of a filler, such as having less than 0.1% by weight, less than 0.05% by weight, less than 0.01% by weight, or non-detectable amount, of a filler. In some embodiments, the pharmaceutical composition herein can also be free or substantially free of a chelating agent, such as having less than 0.1% by weight, less than 0.05% by weight, less than 0.01% by weight, or non-detectable amount, of a chelating agent.

In some embodiments, the pharmaceutical composition is in the form of a unit dosage form.

### Method of Preparation

The pharmaceutical compositions can be prepared by those skilled in the art in view of the present disclosure. In some preferred embodiments, the method of preparation can include a step of freeze drying a therapeutic agent, an aliphatic acid of Formula I described herein or pharmaceutically acceptable salt thereof, and a compound of Formula II described herein or pharmaceutically acceptable salt thereof, which can be beneficial in achieving a superior PK profile when compared to simple blending of these ingredients together, as shown in the Examples section.

In some embodiments, the present disclosure also provides a method of preparing a pharmaceutical composition comprising a therapeutic agent, which comprises: (a) mixing the therapeutic agent (e.g., any of those described herein, such as a polypeptide described herein) with a compound of Formula II described herein or pharmaceutically acceptable salt thereof, and an aliphatic acid of Formula I described herein or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

In some embodiments, the therapeutic agent is mixed first with the compound of Formula II described herein or pharmaceutically acceptable salt thereof, followed by addition of the aliphatic acid of Formula I described herein or pharmaceutically acceptable salt thereof to form the mixture.

In some embodiments, the present disclosure also provide a method of preparing a composition comprising a polypeptide (e.g., any of those described herein, such as semaglutide), the method comprising:
(a) mixing the polypeptide with a compound of Formula II:

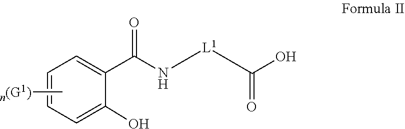

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄alkyl, or C₁₋₄ alkoxy (e.g., OCH₃); and
L¹ is a substituted or unsubstituted C₂-C₁₆ alkylene, or substituted or unsubstituted C₂-C₁₆ alkenylene; and
(b) freeze-drying the mixture formed in (a).

In some embodiments, the mixing in (a) further comprises mixing the polypeptide, compound of Formula II or pharmaceutically acceptable salt thereof, and an aliphatic acid of Formula I: RCOOH, wherein R represents an aliphatic group, or a pharmaceutically acceptable salt thereof. In some embodiments, the aliphatic acid of Formula I is a linear aliphatic acid having 2 to 20 carbon atoms, such as caprylic acid, capric acid, or lauric acid. In some embodiments, the weight ratio of (i) the aliphatic acid of Formula I or pharmaceutically acceptable salt thereof to (ii) the compound of Formula II or pharmaceutically acceptable salt thereof, (i)/(ii), ranges from about 20:1 to about 1:20, such as 5:1 to 1:5, such as about 3:1, about 2:1, about 1:1, about 1:1.5, about 1:2, about 1:2.5, or about 1:3, or any ranges between the recited values, e.g., about 1:2. In some embodiments, the aliphatic acid of Formula I or pharmaceutically acceptable salt thereof is in an amount of about 50 mg to about 300 mg. In some embodiments, the compound of Formula II or pharmaceutically acceptable salt thereof is in an amount of about 200 mg to about 400 mg.

In some specific embodiments, in the methods above as applicable, (a) the therapeutic agent (e.g., any of those described herein) is in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the therapeutic agent (e.g., any of those described herein) is in an amount of about 1 mg to about 200 mg (e.g., about 10 mg about 50 mug, about 100 mg, or any range between the recited values); (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol).

In some specific embodiments, in the methods above as applicable, (a) the therapeutic agent (e.g., any of those described herein) is a polypeptide described herein, for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the therapeutic agent (e.g., any of those described herein) is a polypeptide described herein, for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol).

In some specific embodiments, in the methods above as applicable, (a) the therapeutic agent is a GLP-1 agonist (e.g., any of those described herein), for example, in an amount of about 1 mg to about 200 ng (e.g., about 10 mg, about 50 ng, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the therapeutic agent is a GLP-1 agonist (e.g., any of those described herein), for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mol, 1.3 mmol, 2 mmol, or any range between the recited values, such as 0.9-1.3 mmol).

In some specific embodiments, in the methods above as applicable, (a) the therapeutic agent is semaglutide, for example, in an amount of about 1 mg to about 200 mg (e.g., about 10 mg, about 50 mug, about 100 mg, or any range between the recited values), or in an amount of about 0.1 micromole to about 2.5 micromole, such as about 0.5 micromole to about 2.5 micromole; (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the therapeutic agent is semaglutide, for example, in an amount of about 1 mg to about 200 mg (e.g., about 10 mg, about 50 mg, about 100 mg, or any range between the recited values), or in an amount of about 0.1 micromole to about 2.5 micromole, such as about 0.5 micromole to about 2.5 micromole; (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol).

The composition comprising the therapeutic agent prepared by the method herein is also a novel composition of the present disclosure.

In some embodiments, the present disclosure further provides a method of preparing a pharmaceutical composition comprising mixing the composition comprising the therapeutic agent prepared by the method herein with a pharmaceutically acceptable excipient (e.g., any of those described herein).

### Method of Treatment

The pharmaceutical compositions described herein can be useful for treating a disease or disorder in a subject in need thereof, wherein the disease or disorder can be any of those known to be treatable with the therapeutic agent disclosed herein. The enhanced oral delivery of therapeutic agents as shown in the present disclosure can offer alternative and advantageous treatment options using these therapeutic agents.

For example, in some embodiments, the present disclosure provides a method of treating type-2 diabetes or obesity, in a subject in need thereof, the method comprising orally administering the pharmaceutical composition described herein to deliver a therapeutically effective amount of the therapeutic agent (e.g., GLP-1 agonist described herein) to the subject.

Type 2 diabetes (T2D) is a serious global public health issue, with huge burdens associated with complications resulted from the microvascular and macrovascular diseases. The pathogenesis of diabetes comprises changes in multiple organs, typically with elevated glycemic levels and loss or reduction of the glycemic control. The glycemic control for T2D with different mechanisms of actions has been demonstrated in reduced incidences of microvascular diseases, such as diabetic kidney diseases and diabetic retinopathy. (Chatterjee S, Khunti K, Davies M J. Type 2 diabetes. Lancet. 2017; 389 (10085):2239-2251.).

Incretins are an important class of medications for treatment of diabetes and obesity, including GLP-1, GIP, PYY, etc. GLP-1 receptor agonists have become an important and essential medications that are widely prescribed. GLP-1 is mainly expressed in intestinal L cells and brainstem. The GLP-1 receptor (GLP-1R), a G protein-coupled receptor, is expressed in a variety of tissues, including pancreatic islets, gastrointestinal tract, lung, cardiovascular system, kidney, nodose ganglion neurons of the vagal nerve, the hypothalamus and brainstem in the CNS (Thorens B. Expression cloning of the pancreatic beta cell receptor for the gluco-incretin hormone glucagon-like peptide 1. Proc Natl Acad Sci USA. 1992; 89(18):8641-8645.). GLP-1 expressed from intestinal L cells can circulate and directly bind onto canonical receptors in the pancreatic islet or may indirectly signal the hepatic vagal branch within intraportal vein, potentiating glucose-induced insulin secretion and most postprandial insulin secretion (Pais R, Gribble F M, Reimann F. Stimulation of incretin secreting cells. Ther Adv Endocrinol Metab. 2016; 7(1):24-42.). The signals are sent to the hypothalamus for reducing appetite, stimulating gluconeogenesis, lowering hepatic glucose output, amplifying glucose-dependent insulin release, inhibiting glucagon release, increasing cardiac output and cardioprotection, and decreasing high blood pressure (Muller T D, Finan B, Bloom S R, et al. Glucagon-like peptide 1 (GLP-1). Mol Metab. 2019; 30:72-130.). The function of incretin axis is impaired in T2D with insufficient GLP-1 production, or disrupted GLP-1 action. Therefore, GLP-1 and GLP-1 analogues have been developed as medications for treatment of T2D (Aulinger B A, Vahl T P, Prigeon R L, D'Alessio D A, Elder D A. The incretin effect in obese adolescents with and without type 2 diabetes: impaired or intact? Am J Physiol Endocrinol Metab. 2016; 310(9):E774-781).

Natural GLP-1 is rapidly degraded by dipeptidyl peptidase-IV (DPP-IV) with a half-life at about less than 2 minutes. Therefore, many GLP-1 receptor agonist analogues (GLP-1 RA) were developed with the attempts of prolonging the half-life. Such GLP-1 analogues include: Exendin-4, liraglutide, dulaglutide, lixisenatide, semaglutide, that are approved by US Food and Drug Administration (FDA) for management of T2D. Exendin-4 is a 53% homologous peptide extracted from the venom of a Gila monster. It is resistant to degradation by the DPP4. Structural modifications such as replacement of certain amino acids and/or additions of certain fatty acids were applied to prolong the half-life, allowing once weekly administration from daily administration of GLP-1 analogues; including dulaglutide, albiglutide, liraglutide, lixisenatide, semaglutide (Romera I, Cebria'n-Cuenca A, A'lvarez-Guisasola F, Gomez-Peralta F, Reviriego J. A review of practical issues on the use of glucagon-like peptide-1 receptor agonists for the management of type 2 diabetes. Diabetes Ther. 2019; 10(1):5-19.).

The class of GLP-1 RAs in T2D has demonstrated significant reductions in A1C and a favorable effect on weight control with minimal risk of hypoglycemia (Trujillo J M. Glucagon-like peptide-1 receptor agonists. In: White J R (ed.) Guide to medications for the treatment of diabetes mellitus. Arlington County, VA: American Diabetes Association, 2020, pp. 190-210.). In addition, three of the GLP-1 RAs have demonstrated cardiovascular benefits; dulaglutide, liraglutide, and semaglutide (Matza L S, Boye K S, Sterward D K, et al. Crossover clinical trial assessing patient preference between the dulaglutide pen and the semaglutide pen (PREFER). Diabetes Met Obes 2020; 22: 355-364). The use of GLP-1 RAs is associated with the adverse effects, mainly GI AEs and also injection-site related AEs. The use of GLP-RAs may be also limited by the injection delivery route, resulting in adherence issues. Evaluating the head-to-head studies showed that the long-acting agents result in greater A1C lowering than the short-acting agents, with semaglutide leading to the greatest A1C reduction. Out of the long-acting agents, exenatide XR appears to have the least impact on A1C, although it still produces more A1C lowering compared with the short-acting agents. In regards to weight, there is more ambiguity with the differentiation between agents. The long-acting agents tend to produce more significant weight loss compared with the short-acting agents, with semaglutide once again taking the lead on the greatest weight reduction (Pratley R E, Aroda V R, Lingvay I, et al. Semaglutide versus dulaglutide once weekly in patients with type 2 diabetes (SUSTAIN 7): a randomised, open-label, phase 3b trial. Lancet Diabetes Endocrinol 2018; 6: 275-286.) (Pratley R, Amod A, Hoff S T, et al. Oral semaglutide versus subcutaneous liraglutide and placebo in type 2 diabetes (PIONEER 4): a randomised, double-blind, phase 3a trial. Lancet 2019; 394: 39-50.).GI adverse effects appear to be highest with the short-acting agents as well as subcutaneous semaglutide and appear to be lowest with exenatide XR. Injection site reactions may be more common with the longer acting agents, particularly exenatide once-weekly, which can cause transient small nodules at the injection site. Patient satisfaction data indicate that once weekly injections result in higher patient satisfaction compared with twice daily injections. Discontinuation rates due to adverse events vary between agents and studies, but are low overall with less than 10% of patients in the studies discontinuing GLP-1 RA therapy due to adverse events (Wilke T, Mueller S, Groth A, et al. Nonpersistence and non-adherence of patients with type 2 diabetes mellitus in therapy with GLP-1 receptor agonists: a retrospective analysis. Diabetes Ther 2016; 7: 105-124.). The risk of hypoglycemia is low with GLP-1 RAs and rates were similar across all GLP-1 RA treatment groups. Importantly, current guidelines prioritize the use of GLP-1 RAs with demonstrated CV benefit (dulaglutide, liraglutide, and semaglutide) in patients with atherosclerotic CV disease (ASCVD) and ASCVD risk, independent of baseline A1C.

GLP-1 and GLP-1 analogues are peptides, that have high molecular weight with very low permeability across biological membranes, labile to gut enzymatical degradation, therefore, oral delivery of GLP-1 analogues are typically with very low oral bioavailability. Therefore, all GLP RA therapies are injectables and result in difficultly to use and fear of needles, thus acceptance and adherence of the therapies (Wilke T, Mueller S, Groth A, et al. Nonpersistence and non-adherence of patients with type 2 diabetes mellitus in therapy with GLP-1 receptor agonists: a retrospective analysis. Diabetes Ther 2016; 7: 105-124.).

The first GLP-1 analogue with oral delivery was semaglutide, which was coformulated with an absorption enhancer, sodium N-(8-[2-hydroxybenzoyl]amino) caprylate (SNAC) (Davies M, Pieber T R, Hartoft-Nielsen M L, Hansen O K H, Jabbour S, Rosenstock J. Effect of oral semaglutide compared with placebo and subcutaneous semaglutide on glycemic control in patients with type 2 diabetes: a randomized clinical trial. JAMA. 2017; 318(15):1460-70.290). Although the mechanisms of how SNAC enables the oral absorption of semaglutide are still unclear; tentatively, one or multiple of the following MOAs could be involved: Reduction of enzymatical degradation, optimizing the physicochemical properties of the drug for membrane transport, and enhanced transit fluidity of biological membrane. In the PIONEER trials, the oral semaglutide co-formulated with SNAC has shown efficacy and safety inpatients and the product (Rybelsus®) received its FDA approval on 20 Sep. 2019 (Bucheit J, Pamulapati L G, Carter N, Malloy K, Dixon D L, Sisson E M. Oral semaglutide: a review of the first oral glucagon-like peptide-1 receptor agonist. Diabetes Technol Ther. 2020; 1:10-8). However, while SNAC was shown to enhance the absorption of semaglutide, the oral bioavailability (BA %) was still very low, about 0.5-1% in humans (FDA Clinical Pharmacology Review; https://www.accessdata.fda.gov/drugsatfda_docs/nda/2019/213051Orig1s000ClinPharmR.pdf).

The low bioavailability contributes to high variability in drug exposure in the systemic circulation. In addition, the low bioavailability can significantly increase cost that can become prohibitive to payors, particularly as higher doses of the drug will be needed to control obesity. As an example in the case of semaglutide, the injectable version of semaglutide (Ozempic) has once weekly dosage at 0.5 to 1 mg for diabetic control; while the weekly dosage of 2.4 mg (approved as Wegovy by FDA in 2021) is needed for obesity control. In the oral version of semaglutide (Rybelsus), the daily dose is up to 14 mg (FDA label for Rybelsus and Wegovy). The dose in the oral version needed for obesity may be up to over 50 mg. Chemical or semi-biosynthesis of semaglutide as a modified peptide is costly; currently the cost of semaglutide as an active product ingredient (API) is around US dollar 0.8 per milligram (mg). Therefore, for daily 14 mg of Rybelsus for diabetes indication, the cost of goods (COGs) is around US dollar 336 per month, which is almost approaching the cost of commercial product (5770 per month as priced by Novo Nodisk) https://www.goodrx.com/rybelsus. For daily 50 mg, for potential indications such as obesity, the COGs is about USD 1200 per month and will exceed the acceptable pricing for the payers. Therefore, it is critical and essential to develop an oral formulation of semaglutide with improved BA %, reduced variability, and control the COGs to allow extension of treatment for obesity, fulfilling the medical values of GLP-1 therapeutics and benefiting healthcare of patients with T2D and obesity, as well as reducing the financial burden of healthcare systems.

As discussed herein, the combination of the aliphatic acid of Formula I and the compound of Formula II, or their respective salts, more particularly, sodium caprate and SNAC, achieved a significantly higher oral bioavailability of GLP-1 agonist (in particular semaglutide) compared to using just SNAC as enhancer. Thus, the method herein can advantageously use the pharmaceutical composition herein to orally administer GLP-1 agonist for the treatment of various diseases or disorders for which a GLP-1 agonist can be beneficial, such as type-2 diabetes or obesity.

### Combination Therapies

The pharmaceutical compositions herein can be used as a monotherapy or in a combination therapy. For example, in some embodiments, the pharmaceutical composition can be a fixed dose combination of two or more active therapeutic agents. Non-limiting combination therapies contemplated include the following.

Combination of GLP-RA and SGLT2 inhibitors: cardiovascular benefits. Sodium-glucose cotransporter (SGLT) proteins function independently of insulin in regulation of glucose. Sodium-glucose cotransporter 1 (SGLT1) proteins are high affinity and low-capacity transporters of glucose and are expressed in the small intestines as well as the proximal tubule of the kidneys. The SGLT1 proteins in the proximal convoluted tubule of the kidneys are responsible for less than 10% of filtered glucose reabsorption. Sodium-glucose cotransporter-2 (SGLT2) proteins are expressed in the proximal convoluted tubule of the kidneys and are responsible for roughly 90% of filtered glucose reabsorption (Scheen A J. Pharmacodynamics, efficacy and safety of sodium-glucose co-transporter type 2 (SGLT2) inhibitors for the treatment of type 2 diabetes mellitus. Drugs. 2015 January; 75(i):33-59.). There are several SGLT2 selective inhibitors approved by FDA. Including canagliflozin, dapagliflozin, and empagliflozin. Of the three FDA approved drugs, empagliflozin has the greatest selectivity for SGLT2 compared to SGLT1, while canagliflozin is the least selective (Shubrook J H, Bokaie B B, Adkins S E. Empagliflozin in the treatment of type 2 diabetes: evidence to date. Drug design, development and therapy. 2015; 9:5793-803.). SGLT2 inhibitors have demonstrated clinically body weight control and antihypertensive benefits. The risk of hypoglycemia with SGLT2 inhibitors is small when compared to insulin and sulfonylureas. (Desouza C V, Gupta N, Patel A. Cardiometabolic Effects of a New Class of Antidiabetic Agents. Clin Ther. 2015 Jun. 1; 37(6):1178-94.)

Recent studies demonstrated that there was no increased risk for major adverse cardiovascular events with GLP-RAs, such as dapagliflozin and empagliflozin. More importantly, studies showed that in T2D patients with high risk of cardiovascular disease events, SGLT2 inhibition demonstrated significant CV benefits, such as a 38% relative risk reduction in death from cardiovascular causes in the empagliflozin group versus the placebo group (Zinman B, Wanner C, Lachin J M, Fitchett D, Bluhmki E, Hantel S, et al. Empagliflozin, Cardiovascular Outcomes, and Mortality in Type 2 Diabetes. The New England journal of medicine. 2015 Nov. 26; 373(22):2117-28. This was a pivotal cardiovascular outcomes trial for empagliflozin.).

GLP-1RAs and SGLT-2 inhibitors showed the evidence to improve clinical outcomes in diabetic patients with cardiovascular diseases. The new T2D pharmacotherapy guidelines have recommended the use of GLP-1RAs for prevention and treatment of obese patients with risks of atherosclerotic cardiovascular diseases, whereas SGLT-2is has been proposed for patients with a risk of chronic heart failure. However, there are no systemic research, especially clinical trials to further evaluate the potential additive or synergistic effects of these two classes of medications for the CV benefits, given both classes of drugs have different modes of mechanisms for treatments of T2D and diabetes.

The key reason of not having robust clinical research on the combinations of GLP-RA and SGLT2i may be explained on the commercial or financial basis. GLP-RA are also injectable peptides daily or weekly (except for oral semaglutide) while SGLT2i are all oral tablets administered daily. It is not feasible to develop a financially supportive combination, since such injectable and oral combinations may not be acceptable to patients in practice, and more importantly, no intellectual properties can be obtained for such direct combinations of two commercially available medications.

Commercially, the only available oral GLP-RA is Rybelsus, which however, has too low B/A %, which makes it economically challenging in combinations. Technically, oral semaglutide is absorbed in stomach facilitated by the carrier, SNAC,, while SGLTis are formulated in tablets with coated film, which target absorption in small intestine, where the absorption area is large and the villi of endothelial cells are abundant, allowing much higher permeability than in stomach.

In some embodiments, the present invention enables the formulations of GLP-RA and SGLTi into the same tablets, which not only have improved oral bioavailability of GLP-RA compared to the formulation with SNAC alone, but also allow absorption of SGLTi from the stomach. Such fixed-dose combination of GLP-RA and SGLTis in one tablet brings significant medical values with convenient use and economic feasibility.

### Definitions

As used herein, the singular form “a” “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, 20 carbohydrates, alkyl groups, acyl groups, esters and the like. An example of a derivative of GLP-1(7-37) is NE/26-((4S)-4-(hexadecanoylamino)-carboxy-butanoyl)[Arg34, Lys26]GLP-1-(7-37).

In some embodiments the term “GLP-1 analogue” as used herein refers to a peptide, or a compound, which is a variant of the human Glucagon-Like Peptide-1 (GLP-1(7-37)). GLP-1(7-37) has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or insertions.

In some embodiments, the GLP-1 agonist exhibits at least 60%, 65%, 70%, 80% or 90% sequence identity to GLP-1(7-37) over the entire length of GLP-1(7-37). As an example of a method for determination of sequence identity between two analogues the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

The term “GLP-1 agonist” as used herein refers to a compound, which fully or partially activates the human GLP-1 receptor. In some embodiments, the GLP-1 agonist is a GLP-1 analogue, optionally comprising one substituent. In some embodiments the GLP-1 agonist is exendin-4, the sequence of which is HGEGTFITSDL SKQMEEEAVR-LFIEWLKNGGPSSGAPPPS (SEQ ID No: 2). In some embodiments the GLP-1 agonist comprises one substituent which is covalently attached to the peptide. In some embodiments the substituent comprises a fatty acid or a fatty diacid. In some embodiments the substituent comprises a C16, C18 or C20 fatty acid. In some embodiments the substituent comprises a C16, C18 or C20 fatty diacid. Examples of GLP-1 RA include but not limited to semaglutide, liraglutide, dulaglutide, lixisenatide, exenatide and others.

In some embodiments, the GLP-1 agonist is selected from one or more of the GLP-1 agonists disclosed in WO93/19175, WO96/29342, WO98/08871, WO99/43707, WO99/43706, WO99/43341, WO99/43708, WO2005/027978, WO2005/058954, WO2005/058958, WO2006/005667, WO2006/037810, WO2006/037811, WO2006/097537, WO2006/097538, WO2008/023050, WO2009/030738, WO2009/030771 and WO2009/030774.

In some embodiments, the GLP-1 agonist is selected from the group consisting of N-epsilon37{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon26{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7, Arg34]GLP-1-(7-37); N-epsilon37{2-[2-(2-{2-[2-((S)-3-carboxy-3-{[1-(19-carboxynonadecanoyl) piperidine-4-carbonyl]amino}propionylamino)ethoxy]ethoxy}acetylamino)ethoxy]ethoxy}acetyl[Aib8,Glu22,Arg26,Arg34,Lys37]GLP-1-(7-37)amide; N-epsilon37-[2-(2-[2-(2-[2-(2-((R)-3-[1-(17-carboxyheptadecanoyl)piperidin-4-ylcarbonylamino]3-carboxypropionylamino)ethoxy)ethoxy]acetylamino)ethoxy]ethoxy)acetyl][,DesaminoHis7, Glu22 Arg26, Arg 34, Phe(m-CF3)28]GLP-1-(7-37)amide; N-epsilon26-[(S)-4-carboxy-4-({trans-4-[(19-carboxynonadecanoylamino)methyl]cyclohexanecarbonyl}amino)butyryl][Aib8,Arg34]GLP-1-(7-37); N-epsilon26-{4-[(S)-4-carboxy-4-({trans-4-[(19-carboxynonadecanoylamino) methyl]cyclohexanecarbonyl}amino)butyrylamino]butyryl}[Aib8,Arg34]GLP-1-(7-37); N-epsilon26-[2-(2-{2-[(S)-4-carboxy-4-({trans-4-[(19-carboxy-nonadecanoylamino) methyl]cyclohexanecarbonil}amino)butyrylamino]ethoxy}ethoxy)acetyl][Aib8,Arg34]GLP-1-(7-37); N-epsilon26-1[2-(2-{2-[2-(2-(2-[(S)-4-carboxy-4-({trans-4-[(19-carboxy-nonadecanoylamino)methyl]cyclohexanecarbonyl amino)butyrylamino]ethoxy}ethoxy)acety lamino]ethoxy}ethoxy) acetyl][Aib8,Arg34]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl] [Aib8,Glu22,Arg26, Arg34,Lys37](GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl] [DesaminoHis7,Glu22, Arg26,Arg34,Lys37]GLP-1-(7-37)amide; N-epsilon37-[2-(2-{2-[2-(2-{2-[(S)-4-carboxy-4-({4-[(trans-19-carboxy-nonadecanoylamino)methyl]cyclohexanecarbonyl}amino) butyrylamino]ethoxy}ethoxy)acetylamino]ethoxy}ethoxy)acetyl][DesaminoHis7,Arg26,Arg34,Lys 37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22,Arg26,Arg 34,Lys37]GLP-1-(7-37); N-epsilon26[2-(2-{2-[2-(2-{2-[(S)-4-carboxy-4-({4-[(19-carboxy-nonadecanoylamino)methyl]cyclohexanecarbonyl}amino)butyrylamino] ethoxy}ethoxy) acetylamino]ethoxy}ethoxy)acetyl[Aib8, Lys 26]GLP-1 (7-37)amide; N-epsilon26 [2-(2-[2-(2-[2-(2-((S)-2-[trans-4-((9-carboxynonadecanoylamino]methyl) cyclohexylcarbonylamino]-4-carboxybutanoylamino)ethoxy)ethoxy]acetylamino) ethoxy]ethoxy)acetyl] [Aib8, Lys26]GLP-1 (7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino]ethoxy}ethoxy)acetyl][DesaminoHis7,Arg 26,Arg34,Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acety lamino]ethoxy}ethoxy)acetyl] [DesaminoHis7,Glu22,Arg26,Glu30,Arg34,Lys37]GLP-1-(7-37); N-epsilon26-[2-(2-{2-[(S)-4-carboxy-4-((S)-4-carboxy-4-{4-[4-(16-(1H-tetrazol-5-yl)-hexadecanoylsulfamoyl)butyrylamino]-butyrylamino}butyrylamino) butyrylamino]ethoxy}ethoxy)acetyl] [Aib8,Arg34](GLP-1-(7-37); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino)}butyrylamino) butyrylamino]ethoxy}ethoxy)acetyl] [Aib8,Arg34]GLP-1-(7-37); N-epsilon26-[2-(2-{2-[(S)-4-carboxy-4-((S)-4-carboxy-4-{6-[4-(16-(11-tetrazol-5-yl)hexadecanoyl-sulfamoyl)butyrylamino]hexanoylamino}butyrylamino)butyrylamino]ethoxy}ethoxy) acetyl][Aib8,Arg34]GLP-1-(7-37); N-epsilon26-[2-(2-{2-[(S)-4-carboxy-4-((S)-4-carboxy-4-{4-[4-(16-(1H-tetrazol-5-yl)hexadecanoylsulfamoyl)butyrylamino]butyrylamino}butyrylamino)butyrylamino]ethoxy}ethoxy)acetyl] [Aib8,Arg34]GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]-dodecanoylamino}butyrylamino) butyrylamino] ethoxy}ethoxy)acetyl] [Aib8,Arg34]GLP-1-(7-34); N-epsilon26-[2-(2-2-[(S)-4-carboxy-4-((S)-4-carboxy-4-{6-[4-{6-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-34); N-epsilon26-[2-(2-{2-[(S)-4-carboxy-4-((S)-4-carboxy-4-{12-[4(16-(I1H-tetrazol-5-yl)hexadecanoyl-sulfamoyl)butyrylamino]dodecanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carb oxy-4-{6-[4-(16-(1H-tetrazol-5-yl)hexadecanoylsulfamoyl)butyrylamino]hexanoylamino}butyrylamino)butyrylamino]ethoxy}ethoxy)acetyl][Aib8,Arg34]GLP-1-(7-36)amide; N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino}butyrylamino) butyrylamino]ethoxy}ethoxy)acetyl][Aib8,Arg34],GLP-1-(7-35); N-epsilon26-[2-(2-{2-[(S)-4-carboxy-4-((S)-4-carboxy-4-{2-[4-(16-(1H-tetrazol-5-yl)hexadecanoylsulfamoyl)butyrylamino]dodecanoylamino}butyryl-amino)butyrylamino]ethoxy}ethoxy)acetyl][Aib8,Lys33, Arg34](GLP-1-(7-34); N-epsilon26-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino)butyrylamino]etho xy}ethoxy)acetyl][Aib8,Arg34](GLP-1-(7-36)amide; N-epsilon26-[2-(2-{2-[2-(2-{2-[2-(2-{2-[2-(2-{2-[2-(2-{2-[2-(2-{2-[(S)-4-carboxy-4-((S)-4-carboxy-4-{12-[4-(16-(1 H-tetrazol-5-yl)hexadecanoylsulfamoyl) butyrylamino]dodecanoylamino}butyrylamino) butyrylamino]ethoxy}ethoxy) acetylamino]ethoxy}ethoxy)acetylamino]ethoxy}ethoxy)acetylamino]ethoxy}ethoxy)acetyla mino]ethoxy}ethoxy)acetylamino]ethoxy}ethoxy)acetyl] [Aib8,Lys26,Arg34]GLP-1-(7-36)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][Aib8,Glu22,Arg26,Arg34,Lys37]GLP-1-(7-37)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino]dodecanoylamino}butyrylamino) butyrylamino]ethoxy}ethoxy)acetyl][DesaminoHis7,Glu22,Arg26,Arg34,Lys37]GLP-1-(7-37)amide; N-epsilon37{2-[2-(2-{2-[2-((R)-3-carboxy-3-{[1-(19-carboxy-nonadecanoyl)piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon37{2-[2-(2-{2-[2-((S)-3-carboxy-3-{[1-(19-carboxynonadecanoyl)piperidine-4-carbonyl]amino}propionylamino) ethoxy]ethoxy}acetylamino)ethoxy]ethoxy}acetyl [Aib8,Glu22, Arg26,Arg34, Lys37]GLP-1-(7-37)amide; N-epsilon37-[2-(2-[2-(2-[2-(2-((R)-3-[1-(17-carboxyhepta-decanoyl)piperidin-4-ylcarbonylamino]3-carboxy-propionylamino)ethoxy)ethoxy]acetylamino) ethoxy]ethoxy)acetyl] [DesaminoHis7, Glu22,Arg26, Arg34,Phe(m-CF3)28]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy)acetylamino] ethoxy}ethoxy)acetyl][Aib8,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}ethoxy) acetylamino]ethoxy}ethoxy)acetyl][DesaminoHis7, Glu22,Arg26,Arg34,Lys37]GLP-1-(7-37)amide; N-epsilon 37-[2-(2-{2-[2-(2-{2-[(S)-4-carboxy-4-({trans-4-[(19-carboxy-nonadecanoylamino)methyl]cyclohexanecarbonyl}amino)butyrylamino]ethoxy}eth oxy)acetylamino]ethoxy}ethoxy)acetyl][DesaminoHis7,Glu22,Arg26,Arg34, Lys37]GLP-1-(7-37); N-epsilon37-[2-(2-{2-[2-(2-{2-[(S)-4-carboxy-4-({trans-4-[(19-carboxy-nonadecanoylamino) methyl]cyclohexanecarbonyl}amino)butyrylamino]ethoxy}ethoxy)acetylamino]ethoxy}ethoxy)acetyl][DesaminoHis7,Glu22, Arg26,Glu30, Arg34, Lys37]GLP-1-(7-37); N-epsilon37-[2-(2-{2-[(S)-4-carboxy-4-((S)-4-carboxy-4-{12-[4-(16-(1H-tetrazol-5-yl)hexadecanoylsulfamoyl) butyrylamino]dodecanoylamino} butyrylamino) butyrylamino]ethoxy}ethoxy)acetyl] [Aib8,Glu22,Arg26,Arg34,Lys37]GLP-1-(7-37)amide; N-epsilon37-[2-2-{2-[(S)-4-carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy)acetyl] [DesaminoHis7,Glu22,Arg26,Arg34,Lys37]GLP-1-(7-37)amide; N-epsilon37-(3-((2-(2-(2-(2-(2-Hexadecyloxyethoxy)ethoxy)ethoxy) ethoxy) ethoxy)) propionyl)[DesaminoHis7,Glu22,Arg26,Arg34,Lys37]GLP-1(7-37)-amide; N-epsilon37-{2-(2-(2-(2-[2-(2-(4-(hexadecanoylamino)-4-carboxybutyryl-amino)ethoxy) ethoxy]acetyl)ethoxy)ethoxy)acetyl)}-[desaminoHis7,Glu22,Arg26, Glu30,Arg34,Lys37]GLP-1-(7-37)amide; N-epsilon37-{2-(2-(2-(2-[2-(2-(4-(hexadecanoylamino)-4-carboxybutyryl-amino) ethoxy)ethoxy]acetyl)ethoxy)ethoxy) acetyl)}-[desaminoHis7,Glu22, Arg26, Arg34,Lys37]GLP-1-(7-37)amide; N-epsilon37-(2-(2-(2-(2-(2-(2-(2-(2-(2-(octadecanoyl-amino)ethoxy)ethoxy) acetylamino)ethoxy) ethoxy)acetylamino) ethoxy)ethoxy) acetyl)[desaminoHis7,Glu22,Arg26,Arg34,Lys37]GLP-1 (7-37)amide; N-epsilon37-[4-(16-(1H-Tetrazol-5-yl)hexadecanoylsulfamoyl)butyryl][DesaminoHis7,Glu22,Arg26, Arg34, Lys37]GLP-1-(7-37)amide; N-epsilon37-[2-(2-{2-[2-(2-{2-[(S)-4-carboxy-4-(I 9-carboxynonadecanoylamino) butyrylamino] ethoxy}ethoxy) acetylamino]ethoxy}ethoxy)acetyl] [DesaminoHis7,Glu22,Arg26, Arg34,Lys37]GLP-1-(7-37); N-epsilon37-(2-{2-[2-((S)-4-carboxy-4-{(S)-4-carboxy-4-[(S)-4-carboxy-4-(19-carboxy-nonadecanoylamino)butyrylamino]butyrylamino}butyrylamino)ethoxy]ethoxy}acetyl)[DesaminoHis7,Glu22,Arg26,Arg34,Lys37]GLP-1-(7-37); N-epsilon37-{2-[2-(2-{(S)-4-[(S)-4-(12-{4-[16-(2-tert-Butyl-2H-tetrazol-5-yl)-hexadecanoylsulfamoyl]butyrylamino}dodecanoylamino)-4-carboxybutyrylamino]-4-carboxybutyrylamino}ethoxy)ethoxy]acetyl}[DesaminoHis7,Glu22,Arg26,Arg34,Lys37]GLP-1 (7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][Aib8,Glu22, Arg26,Arg34,Lys37]GLP-1-(7-37); N-alpha37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][Aib8,Glu22,Arg26,Arg34,epsilon-Lys37]GLP-1-(7-37)peptide; N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl][desaminoHis7, Glu22,Arg26,Arg34,Lys37]GLP-1-(7-37); N-epsilon36-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl][desaminoHis7, Glu22,Arg26,Glu30,Arg34,Lys36]GLP-1-(7-37)-Glu-Lys peptide; N-epsilon37-[2-(2-{2-[2-(2-{2-[(S)-4-carboxy-4-({trans-4-[(19-carboxynonadecanoylamino)methyl]cyclohexanecarbonyl}amino)butyrylamino]ethoxy}etho xy)acetylamino]ethoxy}ethoxy)acetyl] [Aib8,Glu22,Arg26,Arg34,Lys37]GLP-1-(7-37); N-epsilon37-[2-(2-{2-[2-(2-{2-[(S)-4-carboxy-4-(17-carboxyheptadecanoylamino)-butyrylamino]-ethoxy}-ethoxy)-acetylamino]-ethoxy}-ethoxy)-acetyl]-[Aib8,Glu22, Arg26,Arg34,Aib35,Lys37]GLP-1-(7-37); N-epsilon37-[(S)-4-carboxy-4-(2-{2-[2-(2-{2-[2-(17-carboxyheptadecanoylamino) ethoxy]ethoxy}acetylamino) ethoxy]ethoxy}acetylamino)butyl] [Aib8,Glu22,Arg26,34,Lys37]GLP-1 (7-37); N-epsilon37-[2-(2-[2(2-[2-(2-[4-(17-carboxyheptadecanoylamino)-4(S)-carboxybutyrylamino]ethoxy)ethoxy]acetylamino)ethoxy]ethoxy)acetyl][ImPr7,Glu22, Arg26,34,Lys37], GLP-1-(7-37); N-epsilon26-{2-[2-(2-{2-[2-(2-{(S)-4-carboxy-4-[10-(4-carboxyphenoxy)decanoylamino]butyrylamino}ethoxy)ethoxy]acetylamino}ethoxy) ethoxy]acetyl}, N-epsilon37-{2-[2-(2-{2-[2-(2-{(S)-4-carboxy-4-[10-(4-carboxyphenoxy) decanoylamino]butyrylamino}ethoxy)ethoxy]acetylamino}ethoxy) ethoxy]acetyl}-[Aib8,Arg34,Lys37]GLP-1(7-37)-OH; N-epsilon26 (17-carboxyheptadecanoyl)-[Aib8,Arg34]GLP-1-(7-37)-peptide; N-epsilon26-(19-carboxynonadecanoyl)-[Aib8,Arg34]GLP-1-(7-37); N-epsilon26-(4-{[N-(2-carboxyethyl)-N-(15-carboxypentadecanoyl)amino]methyl}benzoyl[Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl][Aib8,Arg34]GLP-1-(7-37); N-epsilon26-[2-(2-[2-(2-[2-(2-[4-(19-carboxynonadecanoylamino)-4(S)-carboxybutyrylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][3-(4-Imidazolyl)Propionyl7,Arg34]GLP-1-(7-37); N-epsilon26-[2-(2-[2-(2-[2-(2-[4-(17-carboxyheptadecanoylamino)-(carboxymethyl-amino)acetylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-3(S)-Sulfopropionylamino]ethoxy)ethoxy]acetylamino)ethoxy]ethoxy)acetyl] [Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Gly8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [Aib8,Arg34]GLP-1-(7-37)-amide; N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [Aib8,Arg34,Pro37]GLP-1-(7-37)amide; Aib8,Lys26(N-epsilon26-{2-(2-(2-(2-[2-(2-(4-(pentadecanoylamino)-4-carboxybutyrylamino)ethoxy)ethoxy]acetyl)ethoxy) ethoxy)acetyl)}), Arg34)GLP-1-(7-37)-OH; N-epsilon26-[2-(2-[2-(2-[2-(2-[4-{[N-(2-carboxyethyl)-N-(17-carboxyheptadecanoyl)amino]methyl}benzoyl)amino]ethoxy) ethoxy]acetylamino)ethoxy]ethoxy)acetyl] [Aib8,Arg34]GLP-1(7-37); N-alpha7-formyl, N-epsilon26-[2-(2-[2-(2-[2-(2-[4-(17-carboxyheptadecanoyl-amino)-4(S)-carboxy-butyrylamino]ethoxy)ethoxy]acetylamino)ethoxy]ethoxy)acetyl][Arg34]GLP-1-(7-37); N-epsilon2626-[2-(2-[2-(2-[2-(2-[4-(17-carboxyheptadecanoylamino)-4(S)-carboxy-butyrylamino]ethoxy)ethoxy]acetylamino)ethoxy]ethoxy)acetyl] [Aib8, Glu22, Arg34]GLP-1-(7-37); N-epsilon26{3-[2-(2-{2-[2-(2-{2-[2-(2-[4-(15-(N—((S)-1,3-dicarboxypropyl) carbamoyl)pentadecanoylamino)-(S)-4-carboxybutyrylamino] ethoxy)ethoxy]ethoxy}ethoxy)ethoxy]ethoxy}ethoxy)ethoxy]propionyl}[Aib8,Arg34]GLP-1-(7-37); N-epsilon26-[2-(2-[2-(2-[2-(2-[4-{[N-(2-carboxyethyl)-N-(17-carboxy-heptadecanoyl)amino]methyl}benzoyl)amino](4(S)-carboxybutyryl-amino)ethoxy) ethoxy]acetylamino)ethoxy]ethoxy)acetyl] [Aib8,Arg34]GLP-1(7-37); N-epsilon26-{(S)-4-carboxy-4-((S)-4-carboxy-4-((S)-4-carboxy-4-((S)-4-carboxy-4-(19-carboxy-nonadecanoylamino)butyrylamino)butyrylamino)butyrylamino) butyrylamino}[Aib8,Arg34]GlP-1-(7-37); N-epsilon26-4-(17-carboxyheptadecanoyl-amino)-4(S)-carboxybutyryl-[Aib8,Arg34]GLP-1-(7-37); N-epsilon26-{3-[2-(2-{2-[2-(2-{2-[2-(2-[4-(17-carboxyheptadecanoylamino)-4(S)-carboxybutyrylamino]ethoxy)ethoxy]ethoxy}ethoxy)ethoxy]ethoxy}ethoxy)ethoxy]propionyl}[Aib8,Arg34]GLP-1-(7-37); N-epsilon26-{2-(2-(2-(2-[2-(2-(4-(17-carboxyheptadecanoylamino)-4-carboxybutyrylamino) ethoxy)ethoxy]acetyl)ethoxy)ethoxy)acetyl)}-[Aib8,22,27,30,35, Arg34,Pro37, Lys26]GLP-1 (7-37)amide; N-epsilon26-[2-(2-[2-[4-(21-carboxyuneicosanoylamino)-4(S)-carboxybutyrylamino]ethoxy]ethoxy)acetyl] [Aib8,Arg34]GLP-1-(7-37); and N-epsilon26-[2-(2-[2-(2-[2-(2-[4-(21-carboxyuneicosanoylamino)-4(S)-carboxybutyrylamino]ethoxy)ethoxy]acetylamino)ethoxy]ethoxy)acetyl] [Aib8,Arg34]GLP-1-(7-37).

The term solid dosage form can refer to a tablet, or a capsule filled with solids, or a capsule filled with a solution.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., semaglutide) sufficient to result in amelioration of one or more symptoms of a disorder or condition (e.g. Type 2 Diabetes), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

### EXAMPLES

### Example 1

This example shows that the combination of semaglutide with SNAC and sodium caprate have produced an unexpected higher drug absorption and more effective glucose control than SNAC alone. The effective enhancement based on semaglutide plasma concentration is about 5×. In addition, the superior pharmacological effect of semaglutide based on oral glucose tolerance test (oGTT) was also demonstrated for the combination of semaglutide with SNAC and sodium caprate.

### Preparation of Formulations

Appropriate amounts of drug (e.g., semaglutide sodium) and excipients, such as SNAC or SNAC and sodium caprate (“C10”), and other optional excipients if present, were mixed in water to form a solution. The solution was then freeze dried to provide the formulations.

Representative procedure for preparing formulation containing semaglutide, SNAC, and C10: Weigh semaglutide sodium and add it into a glass vial. Add ˜15 ml of water to dissolve each 65 mg of semaglutide sodium. Then weigh SNAC and add it into above semaglutide water solution. Use a stir bar for stirring. Next add sodium caprate (and other excipients, if necessary), into the solution and stir the solution to make all excipients dissolved. The solution was then freeze-dried.

Formulation 1 prepared according to this procedure contains 10 mg of semaglutide and 300 mg SNAC. Formulation 2 prepared according to this procedure contains 10 mg of semaglutide, 300 mg SNAC, and 150 mg sodium caprate.

The freeze-dried powder was then processed into a tablet form, which was used for the examples herein. The tablet formation was conducted using a manual compressor, a round tablet mold with a diameter of 12 mm, and an are in the surface of the mold.

### Example 2A. Pharmacokinetics Studies and Oral Glucose Tolerance Test

This example describes pharmacokinetics (“PK”) studies of oral semaglutide formulations. In each PK study, oGTT study would also be done in the first day of the experiment.

Dogs were raised in separated cages. Before an experiment, two meals were supplied to the dogs at about 3 p.m. and 7 a.m. every day. The foods are fodder with water in a bowl. In the day before the experiment, after feeding the dogs at 3 p.m., the dogs were fasted overnight for about 18 h. The dogs have access to water overnight, but water was removed at 1 h before dosing. Before dosing, the dogs were put into a sling and then a 0.8 ml of blood was collected from each dog's leg vein using a sterile disposable syringe.

Tablet oral dosing: open the cages and let the dogs climb on the cage by their forelegs. Catch their mouths and open their mouths by hands. Workers will keep a dog's mouth open and put the tablet into the dog's throat using their fingers. Then close the dog's mouth and keep the mouth closed for about ten seconds. The dog would swallow the tablet naturally without any water.

After dosing, 0.8 ml of blood samples were collected into EDTA anticoagulant tubes at the following time points: 15 min, 30 min, 1 h, 2 h, 4 h, 6 h, 8 h, 24 h, 48 h, 72 h, 96 h, 120 h and 168 h from the dog's foreleg vein. After turning upside down for several times for mixing, the blood samples were centrifuged at 3500 rpm for ten minutes. The supernatant plasma samples were removed to clean the tubes and stored at −60° C. in the freezer before measurements. After the collection of the 8 h time point samples, the dogs were released, and food and water were provided to them immediately.

A LC-MS method with MRM mode is used to measure the plasma concentration of semaglutide. 150 μl of acetonitrile with internal standard was added into 50 μl of plasma to precipitate the proteins. After vortex for 30 seconds, the samples were put into sonication for two minutes. The samples were centrifuged at 15400 G for 10 minutes. The supernatant was then removed for analysis. The mass spectrum is AB (Triple Quad 6400+), SHIMADZU. A Sepax Bio-C18(4.6*150 mm, 5 μm) was used. The parameters of the LC-MS are summarized below:
Mobile phase: A: H2O (0.1% Formic acid and 5 mM ammonium acetate; B: ACN (0.2% Formic acid)
Flow rate: 1 ml/min
Column temperature: 40° C.
CAD(psi): 12
Gas1(psi): 60
IS(V): 5500
CUR(psi): 40
Gas2(psi): 55
TEM(° C.):600
Mode: ESI/+
Semaglutide ion pairs: 1029.2 m/z˜136.1 m/z, collision energy is 100V.

As discussed above, in each PK study, oGTT study was also done in the first day of the experiment. After 3.5 h of dosing semaglutide, start the −30 min sampling for glucose level measurement. Blood samples at time t=−15 min and 0 min were collected for glucose measurement. After measuring the background glucose level, a disposable syringe without-needle was used to inject the glucose solution (0.3 mg/ml) to the dog's mouth, based on a dose of 1.5 g glucose/kg. The dog's mouth was then closed to make sure they swallow the solution. This operation might need to be repeated five to six times because only about 10 ml of solution could be dosed to the dogs at one time. After completing the glucose dosing, the glucose concentrations were measured from blood samples using a glucometer at the following time points: t=15 min, 30 min, 45 min, 1 h, 2 h, and 3 h.

The glucometer was purchased from Yuyue Medicals htts://www.yuwell.com/index_en.php/Group/read/id/3). The systemic glucose was measured by collecting approximately 0.05 ml of blood sample from the vein of the dog's foreleg. Then a drop of blood was pushed out to the needle, and let it stay at the tip of the needle for a few seconds. A glucose test strip, inserted in the glucometer in advance, was used to wick the above drop of blood from the tip of the needle. The glucometer then automatically started the measurement, and the results were obtained in approximately 8 seconds later. The measured glucose level was recorded immediately, and the test strip was removed.

### Experimental Results

Experimental results in beagle dogs are summarized for the pharmacokinetics of semaglutide and the corresponding pharmacodynamics from oGTT measurements.

The terminal half-life of semaglutide was determined based on the terminal log-linear phase. The area under the concentration-time (AUC) of semaglutide was calculated using the linear trapezoidal rule. The inter-individual variability was assessed with the coefficient of variation (CV), calculated by the standard deviation divided by the mean.

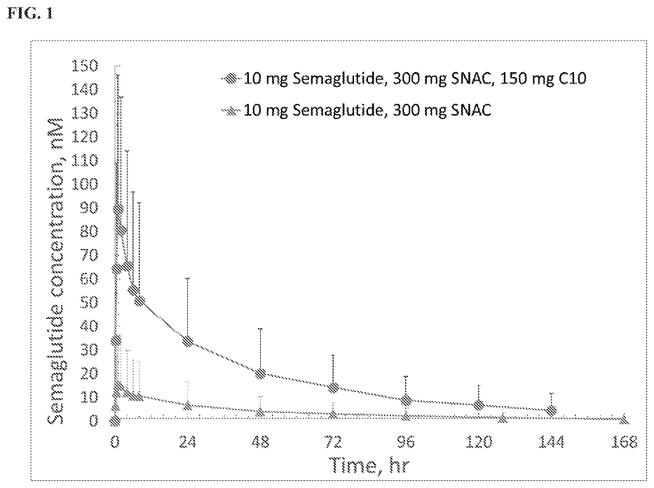

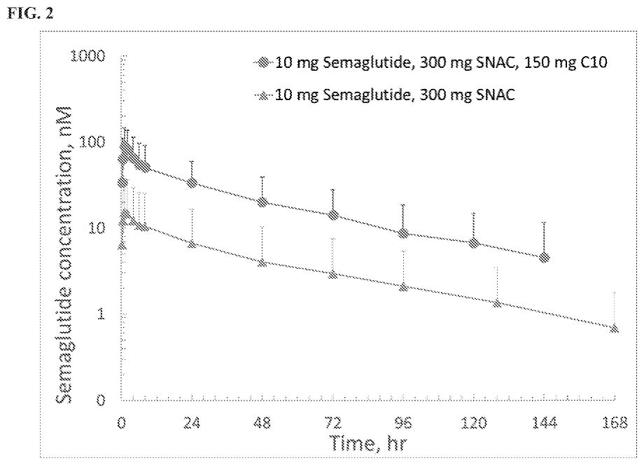

Semaglutide+SNAC In this test, oral formulations containing semaglutide and SNAC in 10 mg and 300 mg, respectively, were administered to dogs as shown above. The concentration-time profiles of semaglutide are shown in FIGS. 1 and 2. The mean pharmacokinetic parameters (N=:6 dogs) are summarized in Table 1. The mean Tmax was at about 1 hr, suggesting rapid absorption facilitated by SNAC as a carrier. The terminal half-life was about 43 hr in dogs. The mean C_max and AUC_infinity was about 16 nM and 647 h·nM, respectively. The corresponding inter-individual variability (IIV) measured by coefficient of variation)

Semaglutide+SNAC+sodium caprate. In this test, oral formulations containing semaglutide, SNAC and sodium caprate in 10 mg, 300 mg and 150 mg, respectively, were administered to dogs as shown above. The concentration-time profiles of semaglutide are shown in FIGS. 1 and 2. The mean pharmacokinetic parameters are summarized in Table 1. The mean Tmax was at about 1 hr, suggesting rapid absorption facilitated by SNAC and sodium caprate as a carrier. The terminal half-life was about 43 hr in dogs. The mean C_max and AUC_infinity was about 90 nM and 3125 nM, respectively.

TABLE 1
Summary of Pharmacokinetic Results

| | Tmax | Cmax | | AUCinf | |
|---|---|---|---|---|---|
| Formulations | hr | nM | CV_Cmax | nM*hr | CV_AUC |
| Semgalutide 10 | 1 | 15.2 | 138 | 647 | 149 |
| mg + SNAC 300 | | | | | |
| mg | | | | | |
| Semaglutide 10 | 1 | 89.6 | 56.4 | 3107 | 83 |
| mg + SNAC 300 | | | | | |
| mg + sodium | | | | | |
| caprate 150 mg | | | | | |

In comparison to the formulation containing SNAC alone, the formulation containing SNAC and sodium caprate showed a substantial improvement of AUC and Cmax, in about 4.8 and 5.9 fold higher, respectively. Also the CV of Cmax and AUC was significantly reduced, which is clinically important, because the smaller inter-individual difference means that medications to the patients are safer (smaller proportion of patients receive unnecessary high exposure) and more efficacious (smaller proportion of patients receive sub-therapeutic exposure).

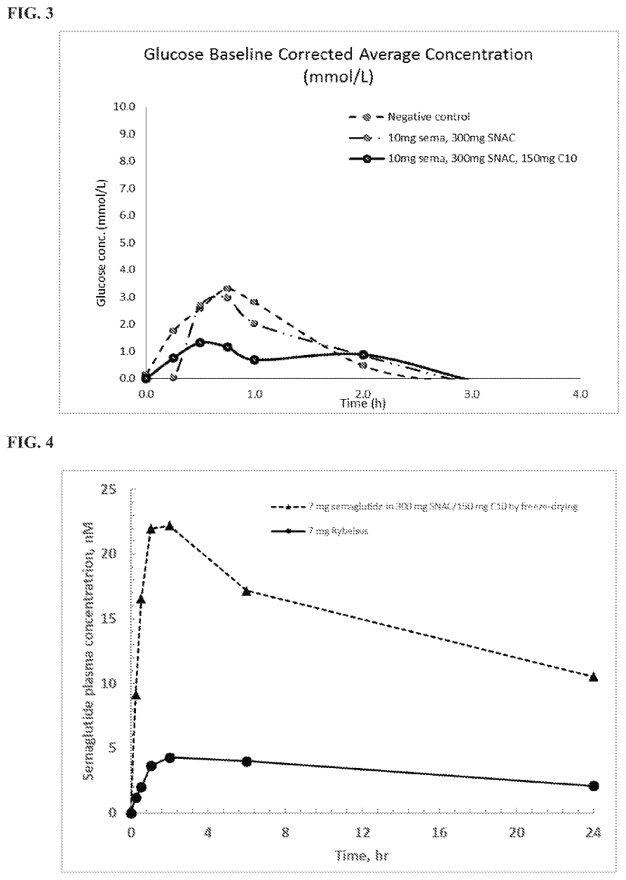

Oral glucose tolerance test (oGTT): The oral dose of glucose used in the study is 1.5 g glucose/kg. The concentration-time profiles of glucose from the formulations containing 10 mg semaglutide with SNAC 300 mg alone, and 10 mg semaglutide with SNAC 300 mg and sodium caprate 150 mg are shown in FIG. 3. oGTT reflects a pharmacodynamic regulation of glucose absorption and disposal by GLP-1 receptor agonist (Courtney Moore, et al., Am J Physiol Endocrinol Metab. 2013 Dec. 15; 305(12): E1473-E1482), 10 mg Semaglutide with SNAC alone showed slight improvement (specifically the early time at 0.25 hr) of glucose control compared to that of control group (no semaglutide). In contrast 10 mg Semaglutide with SNAC plus sodium caprate showed more significant glucose control than that of 10 mg Semaglutide with SNAC alone, indicating that the measured semaglutide concentrations are pharmacodynamically effective, and also consistent with the improved plasma pharmacokinetics from the formulation of SNAC plus sodium caprate.

### Example 3

This example compares (1) the effect of using SNAC alone, C10 alone, or the combination of SNAC and C10 on bioavailability of semaglutide; (2) the effect of using freeze-drying and simple blending on bioavailability of semaglutide; and (3) a representative formulation of this application with a commercial tablet (Rybelsus 7 mg).

### Preparation of Formulation

For samples that use freeze-drying as blend process, the preparation process could be seen below. Appropriate amounts of drug (e.g., semaglutide sodium) and excipients, such as SNAC alone or sodium caprate (“C10”) alone, or SNAC and C10 together, and other optional excipients if present, were mixed in water to form a solution. The solution was then freeze dried to provide the formulations.

Representative procedure for preparing formulation containing semaglutide, SNAC, and C10: Weigh semaglutide sodium and add it into a glass vial. Add about 15 ml of water to dissolve each 65 mg of semaglutide sodium. Then weigh C10 and add it into above semaglutide water solution. Use a stir bar for stirring. Next add SNAC (and other excipients, if necessary), into the solution and stir the solution to make all excipients dissolved. The solution was then freeze-dried and a dry mixed was obtained.

For samples that use simple-bending as blend process, the preparation process could be seen below. Appropriate amounts of drug (e.g., semaglutide sodium) and excipients, such as SNAC and sodium caprate (“C10”), and other optional excipients if present, were mixed in a mortar or mixed in a plastic bag by shaking up and down for at least for 20 min. Then a mixer by simple-blending was obtained.

The powder fabricated by freeze-dried or simple blending was then processed into a tablet form, which was used for the examples herein. The tablet formation was conducted using a manual compressor, with a round tablet mold with a diameter of 10 mm, and an arc in the surface of the mold.

### Pharmacokinetics Studies and LC/MS Assay

This example describes pharmacokinetics (“PK”) studies of oral semaglutide formulations.

Dogs were raised in separated cages. Before an experiment, two meals were supplied to the dogs at about 3 p.m. and 7 a.m. every day. The foods are fodder with water in a bowl. In the day before the experiment, after feeding the dogs at 3 p.m., the dogs were fasted overnight for about 18 h. The dogs have access to water overnight, but water was removed at 1 h before dosing. Before dosing, the dogs were put into a sling and then a 0.8 ml of blood was collected from each dog's leg vein using a sterile disposable syringe.

Tablet oral dosing: open the cages and let the dogs climb on the cage by their forelegs. Catch their mouths and open their mouths by hands. Workers will keep a dog's mouth open and put the tablet into the dog's throat using their fingers. Then close the dog's mouth and keep the mouth closed for about ten seconds. The dog would swallow the tablet naturally without any water.

After dosing, 0.6 ml of blood samples were collected into EDTA anticoagulant tubes at the following time points: pre-dose, 15 min, 30 min, 1 h, 2 h, 4 h, 6 h, 24 h, and 48 h from the dog's foreleg vein. After turning upside down for several times for mixing, the blood samples were centrifuged at 3500 rpm for ten minutes. The supernatant plasma samples were removed to clean the tubes and stored at −60° C. in the freezer before measurements. After the collection of the 6 h time point samples, the dogs were released, and food and water were provided back to them immediately.

A LC-MS method with MRM mode is used to measure the plasma concentration of semaglutide. 150 μl of acetonitrile with internal standard was added into 50 μl of plasma to precipitate the proteins. After vortex for 30 seconds, the samples were put into sonication for two minutes. The samples were centrifuged at 15400 G for 10 minutes. The supernatant was then removed for analysis. The mass spectrum is AB (Triple Quad 6400+), SHIMADZU. A Sepax Bio-C18(4.6*150 mm, 5 mm) was used. The parameters of the LC-MS are summarized below:
Mobile phase: A: H₂O (0.1% Formic acid and 5 mM ammonium acetate; B: ACN (0.2% Formic acid)
Flow rate: 1 ml/min
Column temperature: 40° C.
Mode: ESI/+
Semaglutide ion pairs: 1029.2 m/z˜136.1 m/z.

### Results

As shown in FIG. 4, the use of a combination of SNAC (300 mg)/C10 (150 mg) significantly enhanced semaglutide bioavailability/better PK profile compared to the commercial tablet Rybelsus. The details PK parameters are also shown in the table 2 below, the AUC is enhanced by about 10 fold and Cmax by close to 14 fold.

TABLE 2
Comparison of pharmacokinetics of 7 mg semaglutide formulated
in 300 mg SNAC/150 mg C10 by freeze-drying to that of
7 mg semaglutide of a commercial tablet (Rybelsus)

| 7 mg semaglutide in 300 mg SNAC/150 mg | | | |
|---|---|---|---|
| C10 freeze drying (N = 12) | | Rybelsus (N = 12) | |
| AUC_{0-24 hr}, | | AUC_{0-24 hr}, | |
| nM*hr | Cmax, nM | nM*hr | Cmax, nM |
| (CV %) | (CV %) | (CV %) | (CV %) |
| 186 (103%) | 32.6 (107%) | 18.9 (147%) | 2.39 (156%) |

As shown in FIG. 5, preparing the formulation using freeze drying is beneficial in enhancing semaglutide bioavailability and leads to a much better PK profile, compared to formulations prepared by simple blending. The details PK parameters are also shown in the table 3 below, the AUC is enhanced by about 4 fold and Cmax also by about 4 fold.

TABLE 3
Pharmacokinetics of 10 mg semaglutide formulated in 300 mg
SNAC/150 mg C10 by freeze-drying versus by simple blending

| | |
|---|---|
| 10 mg semaglutide | 10 mg semaglutide |
| in 300 mg SNAC/150 | in 300 mg SNAC/150 |
| mg C10 (N = 12) | mg C10 (N = 5) |
| freeze-drying | simple blending |

| AUC0-24 hr, | | AUC0-24 hr, | |
|---|---|---|---|
| nM*hr | Cmax, nM | nM*hr | Cmax, nM |
| (CV %) | (CV %) | (CV %) | (CV %) |
| 1190 (74%) | 89.9 (63%) | 269 (151%) | 19.5 (152%) |

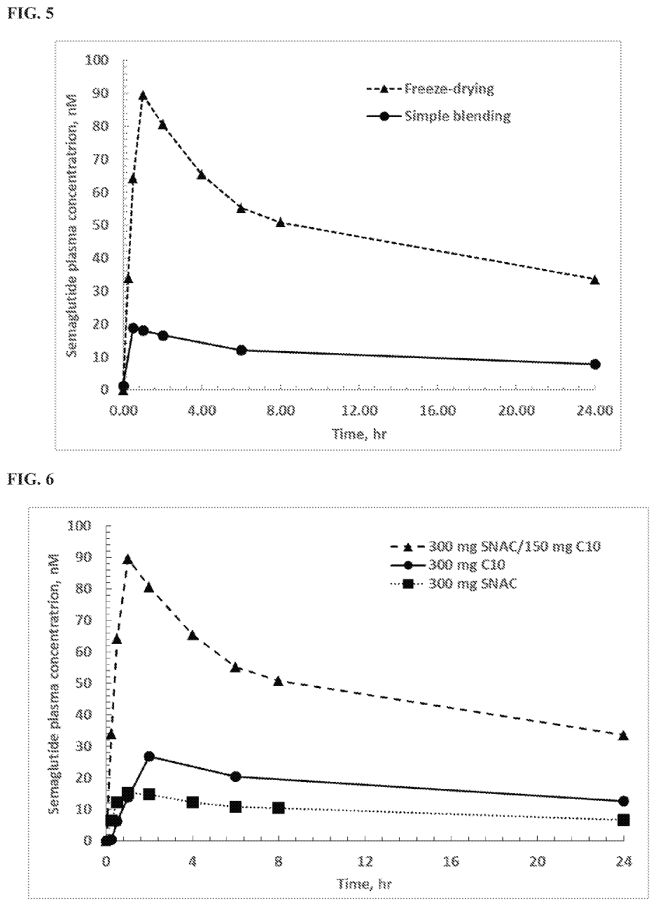

As shown in FIG. 6, the use of SNAC and C10 in combination achieved a synergistic effect and much better PK profiles were achieved when compared to the use of SNAC alone or C10 alone. The details PK parameters are also shown in the table 4 below. As shown in Table 4, the additive effect of 300 mg SNAC and 300 C10 is less than those observed in the combination of SNAC/C10 (even at a lower amount of C10) in both AUC and Cmax.

TABLE 4
Pharmacokinetics of 10 mg semaglutide formulated in 300 mg
SNAC, 300 mg C10, 300 mg SNAC/150 mg C10 by freeze-drying

| 10 mg semaglutide in | | 10 mg semaglutide in | | 10 mg semaglutide in | |
|---|---|---|---|---|---|
| 300 mg SNAC freeze- | | 300 mg C10 freeze- | | 300 mg SNAC/150 mg | |
| drying | | drying | | C10 freeze-drying | |
| AUC_{0-24 hr}, | Cmax, | AUC_{0-24 hr}, | Cmax, | AUC_{0-24 hr}, | Cmax, |
| nM*hr | nM | nM*hr | nM | nM*hr | nM |
| (CV %) | (CV %) | (CV %) | (CV %) | (CV %) | (CV %) |
| 234 | 16.2 | 419 | 27.4 | 1190 | 89.9 |
| (142%) | (134%) | (93%) | (91%) | (74%) | (63%) |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A pharmaceutical composition comprising:
(a) a polypeptide;
(b) an aliphatic acid of Formula I: RCOOH, wherein R represents an aliphatic group, or a pharmaceutically acceptable salt thereof; and
(c) a compound of Formula II: 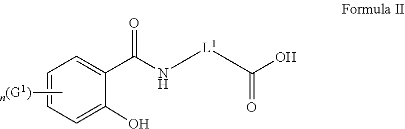
or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

2. The pharmaceutical composition of claim 1, formulated for oral administration.

3. The pharmaceutical composition of claim 1 or 2, which upon oral administration to a human subject in need thereof, delivers a therapeutically effective amount of the polypeptide to the human subject.

4. The pharmaceutical composition of any of claims 1-3, wherein in Formula I, R represents an alkyl group having 1-30 carbon atoms, e.g., R is —(CH₂)₁₋₁₈CH₃.

5. The pharmaceutical composition of any of claims 1-3, wherein in Formula I, R represents an alkyl group having 3-20 carbon atoms.

6. The pharmaceutical composition of any of claims 1-3, wherein in Formula I, R represents an alkyl group having 5-16 carbon atoms.

7. The pharmaceutical composition of any of claims 1-3, wherein the aliphatic acid of Formula I is a linear aliphatic acid having 2 to 20 carbon atoms, such as caprylic acid, capric acid, or lauric acid.

8. The pharmaceutical composition of any of claims 1-7, wherein in Formula II, n is 0.

9. The pharmaceutical composition of any of claims 1-7, wherein in Formula II, n is 1 and G¹ is halogen, C₁₋₄ alkyl, or C₁₋₄ alkoxy.

10. The pharmaceutical composition of any of claims 1-7, wherein in Formula II, n is 1 and G¹ is Cl or OCH₃.

11. The pharmaceutical composition of any of claims 1-10, wherein in Formula II, L¹ is a substituted or unsubstituted C₂-C₁₆ alkylene.

12. The pharmaceutical composition of any of claims 1-10, wherein in Formula II, L¹ is an unsubstituted C₃-C₁₅ alkylene.

13. The pharmaceutical composition of any of claims 1-10, wherein in Formula II, L¹ is an unsubstituted C₅-C₁₃ alkylene.

14. The pharmaceutical composition of any of claims 1-10, wherein in Formula II, L¹ is an unsubstituted, straight-chained C₅-C₉ alkylene.

15. The pharmaceutical composition of any of claims 1-7, wherein the compound of Formula II is

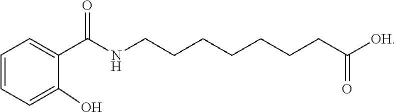 [SMILES: O=C(O)CCCCCCCNC(=O)c1ccccc1O]

16. The pharmaceutical composition of any of claims 1-7, wherein the compound of Formula II is

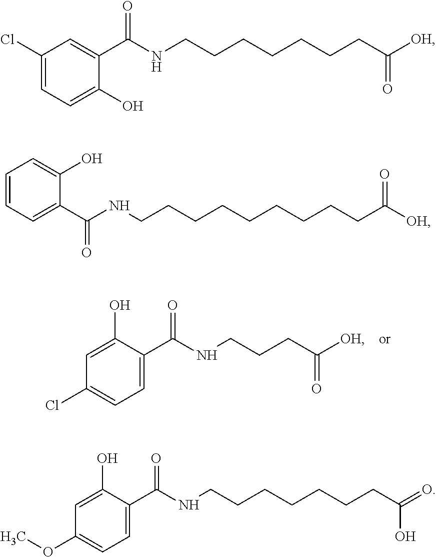 [SMILES: O=C(O)CCCCCCCNC(=O)c1cc(Cl)ccc1O ; O=C(O)CCCCCCCCCNC(=O)c1ccccc1O ; O=C(O)CCCNC(=O)c1ccc(Cl)cc1O ; COc1ccc(C(=O)NCCCCCCCC(=O)O)c(O)c1]

17. The pharmaceutical composition of any of claims 1-16, comprising a sodium salt of the compound of Formula II.

18. The pharmaceutical composition of any of claims 1-3, comprising a sodium salt of

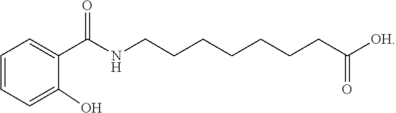 [SMILES: O=C(O)CCCCCCCNC(=O)c1ccccc1O]

19. The pharmaceutical composition of claim 18, wherein the aliphatic acid of Formula I is capric acid.

20. The pharmaceutical composition of any of claims 1-19, wherein the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, such as about 3:1, about 2:1, about 1:1, about 1:1.5, about 1:2, about 1:2.5, or about 1:3, or any ranges between the recited values, e.g., about 1:2.

21. The pharmaceutical composition of any of claims 1-20, wherein the aliphatic acid of Formula I or pharmaceutically acceptable salt thereof is in an amount of about 50 mg to about 300 mg per unit dose.

22. The pharmaceutical composition of any of claims 1-21, wherein the compound of Formula II or pharmaceutically acceptable salt thereof is in an amount of about 200 mg to about 400 mg per unit dose.

23. The pharmaceutical composition of any of claims 1-22, comprising a synergistic combination of (b) the aliphatic acid of Formula I or pharmaceutically acceptable salt thereof and (c) the compound of Formula II or pharmaceutically acceptable salt thereof, for achieving enhanced oral delivery of the polypeptide.

24. The pharmaceutical composition of any of claims 1-23, wherein the polypeptide is a Glucagon-Like Peptide-1 (GLP-1) receptor agonist. (e.g., U.S. Ser. No. 10/960,052).

25. The pharmaceutical composition of any of claims 1-22, wherein the polypeptide is semaglutide, liraglutide, dulaglutide, lixisenatide, or exenatide.

26. The pharmaceutical composition of any of claims 1-25, further comprises a SGLT-2 inhibitor, such as empagliflozin, canagliflozin, dapagliflozin, or ertugliflozin.

27. The pharmaceutical composition of any of claims 1-26, further comprises a DPP-4 inhibitor, such as sitagliptin, vildagliptin, saxagliptin, linagliptin, or alogliptin.

28. The pharmaceutical composition of any of claims 1-27, further comprises insulin.

29. The pharmaceutical composition of any of claims 1-28, further comprising a lubricant, a binder, a filler, and/or a chelating agent (e.g., ethylene diamine tetraacetate (EDTA)).

30. The pharmaceutical composition of any of claims 1-29, in the form of a solid oral dosage form, such as capsule or tablet.

31. A method of treating a disease or disorder, e.g., type-2 diabetes or obesity, in a subject in need thereof, the method comprising orally administering the pharmaceutical composition of any of claims 1-30 to deliver a therapeutically effective amount of the polypeptide to the subject.

32. A method of preparing the pharmaceutical composition of any of claims 1-30, the method comprising (a) mixing the polypeptide with the compound of Formula II or pharmaceutically acceptable salt thereof, and the aliphatic acid of Formula I or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

33. A method of preparing a composition comprising a polypeptide, the method comprising:
(a) mixing the polypeptide with a compound of Formula II: 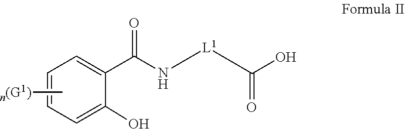
or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₆ alkylene, or substituted or unsubstituted C₂-C₁₆ alkenylene; and
(b) freeze-drying the mixture formed in (a).

34. The method of claim 33, wherein the mixing in (a) comprises mixing the polypeptide with a sodium salt of the compound of Formula II.

35. The method of claim 33 or 34, wherein the mixing in (a) comprises mixing the polypeptide with a sodium salt of

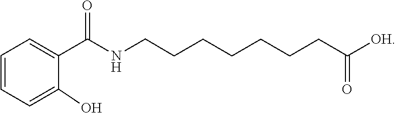 [SMILES: O=C(O)CCCCCCCNC(=O)c1ccccc1O]

36. The method of any one of claims 33-35, wherein the polypeptide is semaglutide, liraglutide, dulaglutide, lixisenatide, or exenatide.

37. The method of any one of claims 33-36, wherein the mixing in (a) further comprises mixing the polypeptide, compound of Formula II or pharmaceutically acceptable salt thereof, and an aliphatic acid of Formula I: RCOOH, wherein R represents an aliphatic group, or a pharmaceutically acceptable salt thereof.

38. The method of claim 37, wherein the aliphatic acid of Formula I is a linear aliphatic acid having 2 to 20 carbon atoms, such as caprylic acid, capric acid, or lauric acid.

39. The method of any of claims 37-38, wherein the weight ratio of (i) the aliphatic acid of Formula I or pharmaceutically acceptable salt thereof to (ii) the compound of Formula II or pharmaceutically acceptable salt thereof, (i)/(ii), ranges from about 20:1 to about 1:20, such as 5:1 to 1:5, such as about 3:1, about 2:1, about 1:1, about 1:1.5, about 1:2, about 1:2.5, or about 1:3, or any ranges between the recited values, e.g., about 1:2.

40. The method of any of claims 37-39, wherein the aliphatic acid of Formula I or pharmaceutically acceptable salt thereof is in an amount of about 50 mg to about 300 mg.

41. The method of any of claims 33-40, wherein the compound of Formula II or pharmaceutically acceptable salt thereof is in an amount of about 200 mg to about 400 mg.

42. The composition prepared by the method of any one of claims 33-41.

43. A method of preparing a pharmaceutical composition comprising mixing the composition of claim 42 with a pharmaceutically acceptable excipient.
